(19) United States
(12) Patent Application Publication
(10) Pub. No.: US 2004/0198743 A1
(43) Pub. Date: Oct. 7, 2004

(54) Methods for treating allergic skin and allergic ocular conditions using combinations of histamine receptor antagonists

(75) Inventors: John A. Hey, Randolph, NJ (US); William Kreutner, West Paterson, NJ (US); Robbie L. McLeod, Branchburg, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/767,164

(22) Filed: Jan. 29, 2004

Related U.S. Application Data
(60) Provisional application No. 60/443,948, filed on Jan. 31, 2003.

(51) Int. Cl. A61K 31/4965; A61K 31/445; A61K 31/495

(52) U.S. Cl. 514/255.04; 514/317

(57) ABSTRACT

The present invention includes methods of treating allergic skin conditions and disorders by combined administration of an histamine H1 receptor antagonist and a histamine H3 receptor antagonist.

This application claims the benefit of U.S. Provisional Patent Application No. 60/443,948, filed Jan. 31, 2003, which is herein incorporated by reference in its entirety.

### FIELD OF THE INVENTION

The present invention relates to methods of treatment of allergic disorders, particularly allergic skin or ocular conditions.

### BACKGROUND OF THE INVENTION

Allergic urticaria is one of the most common allergic dermatological conditions in the U.S. (Ring, et al., (1999) Clin. Exper. Allergy. 29: 31-37). The prevalence of all types of urticaria in the U.S. range up to 35% of the population. The biogenic amine, histamine, is known to be a major contributor to the generation of the wheal and flare skin lesions associated with urticaria. This condition may be caused by release of histamine and other mediators, by skin mast cells, which produce localized vasodilation, increased postcapillary permeability and itching. Second generation, non-sedating antihistamines such as cetirizine and loratadine are current, known treatments of acute urticaria (Monroe, (1993) Ann. Allergy 71: 585-591; Ormerod, (1994) Drug 48: 717-730; Simons, et al., (1994) N. Eng. J. Med. 330: 1663-1670).

The physiological and pathological actions of histamine are mediated through four histamine receptor subtypes: H1, H2, H3 and H4. The erythema, wheal formation and itching associated with urticaria are believed to be mediated by activation of histamine H1 receptors. Histamine H2 receptors may also play a role in the wheal response produced by localized histamine; several investigators have demonstrated that H2 antagonists attenuate the immediate vascular responses of intradermal (i.d.) injections of histamine (Marks, et al., (1977) Br. J. Clin. Pharmacol. 4:364-369; Miller, et al., (1989) J. Allergy Clin. Immunol. 84: 895-899). H4 receptors are found primarily on inflammatory cells, however, a role for the receptor in allergic skin diseases has yet to be defined.

Combination treatment with a histamine H1 receptor antagonist and a histamine H2 receptor antagonist is more effective in reducing the urticaria, itching and wheal and flare responses than treatment with either an H1 or H2 antagonist alone (Phanuphak, et al., (1978) Clin. Allergy 8:429-433; Kaur, et al., (1981) Br. J. Dermatol. 104: 185-190; Monroe, et al., (1981) Arch. Dermatol. 117: 404-407; Mansfield, et al., (1983) Ann. Allergy 50: 241-245 Paul, et al., (1986) Eur. J. Clin. Pharmacol. 31: 277-280; Bleehen, et al., (1987) Br. J. Dermatol. 117: 81-88). However, the synergistic effect of combined histamine H1 receptor antagonist and a histamine H2 receptor antagonist treatment for urticaria remains controversial since some investigators have not been able to demonstrate an improvement in chronic idiopathic urticaria with dual H1 and H2 treatment (Commens, et al., (1978) Br. J. Dermatol. 99: 675-679; Cook, et al., (1993) Acta. Derm. Vanereol. (Stockh) 63:265-267).

Urticaria, commonly known as hives, is a condition characterized by often severe itching which can disrupt an individual's ability to sleep or work. Urticaria is often acute, lasting from a few hours to less than six weeks. Some cases are chronic, lasting more than six weeks. It is a distressing disorder which affects an estimated 20 percent of the population at one time or another in their lives.

In general, histamine H3 receptors are located presynaptically on postganglionic sympathetic noradrenergic nerves, including sympathetics innervating the heart and blood vessels (Imamura, et al., (1995) J. Pharmac. Exp. Ther. 77: 206-210; Li, et al., (1998) J. Appl. Physiol. 85: 1693-1701; Malinowska, et al., (1998) J. Physiol. Pharmacol. 49: 191-211). The presence of functional histamine H3 receptors in the cardiovascular system has been demonstrated in vitro in human, guinea pig, rabbit and rat effector systems, and in vivo in the rat, guinea pig and dog (Malinowska, et al., (1998) J. Physiol. Pharmacol. 49: 191-211). Stimulation of histamine H3 receptors may produce vasodilation by decreasing the release of noradrenaline from noradrenergic nerves terminals. Investigation of the contribution of histamine H3 receptors to skin responses mediated by histamine is an area of great interest and is progressing (Arrang, et al., (1983) Nature 302: 832-837).

Combined administration of a histamine H1 receptor antagonist and a histamine H3 receptor antagonist for treatment of nasal congestion is known (McLeod, et al., (1999) Amer. J. Rhinol. 13: 391-399). In this study, combined blockade of the histamine H1 receptor and the histamine H3 receptor enhanced the efficacy of histamine H1 receptor antagonists in conferring decongestant activity. The ability of the H1/H3 antagonist combination to treat allergic skin conditions was not investigated. McLeod, et al. (Prog. Respir. Res. Basel, Karger (2001) 31: 133-136) also disclose preclinical findings characterizing decongestant activity caused by combination histamine H1 and H3 receptor blockade.

Further, U.S. Pat. No. 5,869,479 discloses compositions for the treatment of the symptoms of allergic rhinitis using a combination of at least one histamine H1 receptor antagonist and at least one histamine H3 receptor antagonist.

Currently, there is a need in the art to characterize the effect of dual histamine H1 receptor and histamine H3 receptor blockade on allergic skin responses which is addressed herein. The present invention surprisingly found that, given together, a histamine H1 receptor antagonist and a histamine H3 receptor antagonist synergistically attenuated allergic skin responses to a greater extent than either an H1 or H3 antagonist alone. Accordingly, the present invention provides treatments for allergic skin conditions, such as urticaria, comprising a histamine H1 receptor antagonist and a histamine H3 receptor antagonist.

### SUMMARY OF THE INVENTION

The present invention provides a method for treating or preventing symptoms of an allergic skin condition (e.g., urticaria) or an allergic ocular condition (e.g., hay fever conjunctivitis, perennial allergic conjunctivitis, giant papillary conjunctivitis, vernal keratoconjunctivitis or atopic keratoconjunctivitis) in a subject (e.g., a human), comprising administering one or more histamine H1 receptor antagonists and one or more histamine H3 receptor antagonists to the subject or comprising administering a single substance which antagonizes both the histamine H1 receptor and the histamine H3 receptor (dual H1/H3 antagonist) to the subject. The antagonists may be administered to the subject along with a pharmaceutically acceptable carrier in a pharmaceutical composition. Preferably, the subject is administered an amount of H1 and H3 antagonist sufficient to produce an anti-histaminic effect. The histamine H1 receptor antagonist and the histamine H3 receptor antagonist may be present in a single dosage form or in separate dosage forms. The antagonists may also be administered along with an additional agent, such as non-steroidal anti-inflammatory drug (NSAID), a steroid or an antibiotic. The present invention further comprises combinations comprising one or more histamine H1 receptor antagonists and one or more histamine H3 receptor antagonists and pharmaceutical compositions thereof.

The histamine H1 receptor antagonist is one or more members selected from the group consisting of astemizole, azatadine, azelastine, acrivastine, brompheniramine, chlorpheniramine, clemastine, cyclizine, carebastine, cyproheptadine, carbinoxamine, desloratadine, doxylamine, diphenhydramine, cetirizine, dimenhydrinate, dimethindene, ebastine, epinastine, efletirizine, fexofenadine, hydroxyzine, ketotifen, loratadine, levocabastine, mizolastine, mequitazine, mianserin, norebastine, meclizine, norastemizole, picumast, pyrilamine, promethazine, terfenadine, tripelennamine, temelastine, trimeprazine, triprolidine and

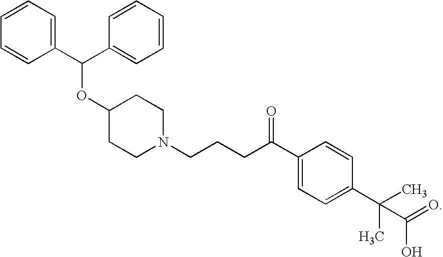

Preferably, the histamine H1 receptor antagonist is one or members selected from the group consisting of loratadine, desloratadine, chlorpheniramine, diphenhydramine, cetirizine, fexofenadine and promethazine.

The histamine H3 receptor antagonist one or more members selected from the group consisting of thioperamide, impromidine, burimamide, clobenpropit, impentamine, mifetidine, clozapine, S-sopromidine, R-sopromidine, ciproxifam, SKF-91486 (3-(imidazole-4-yl)-propylguanidine sulfate), GR-175737 (Clitherow, et al., (1996) Bioorg. Med. 6: 833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50), ABT331440 (PCT Publication No. WO 02/06223),

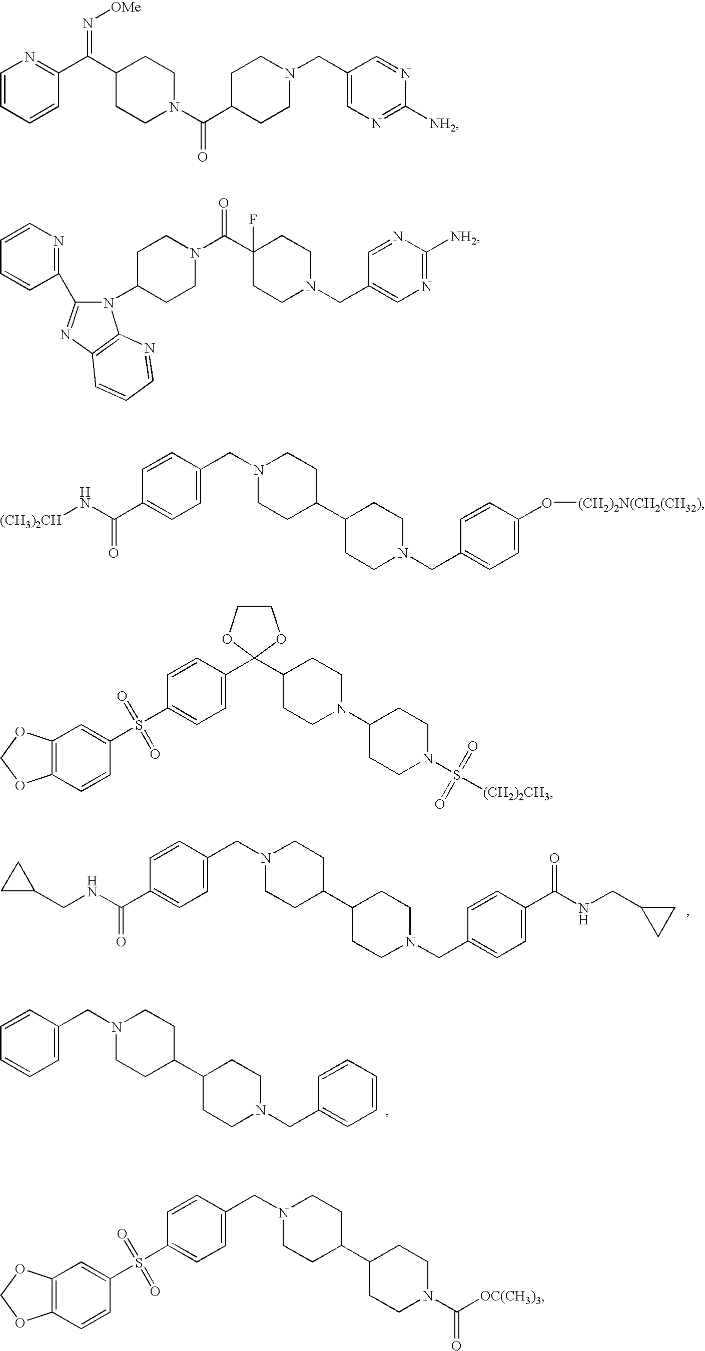

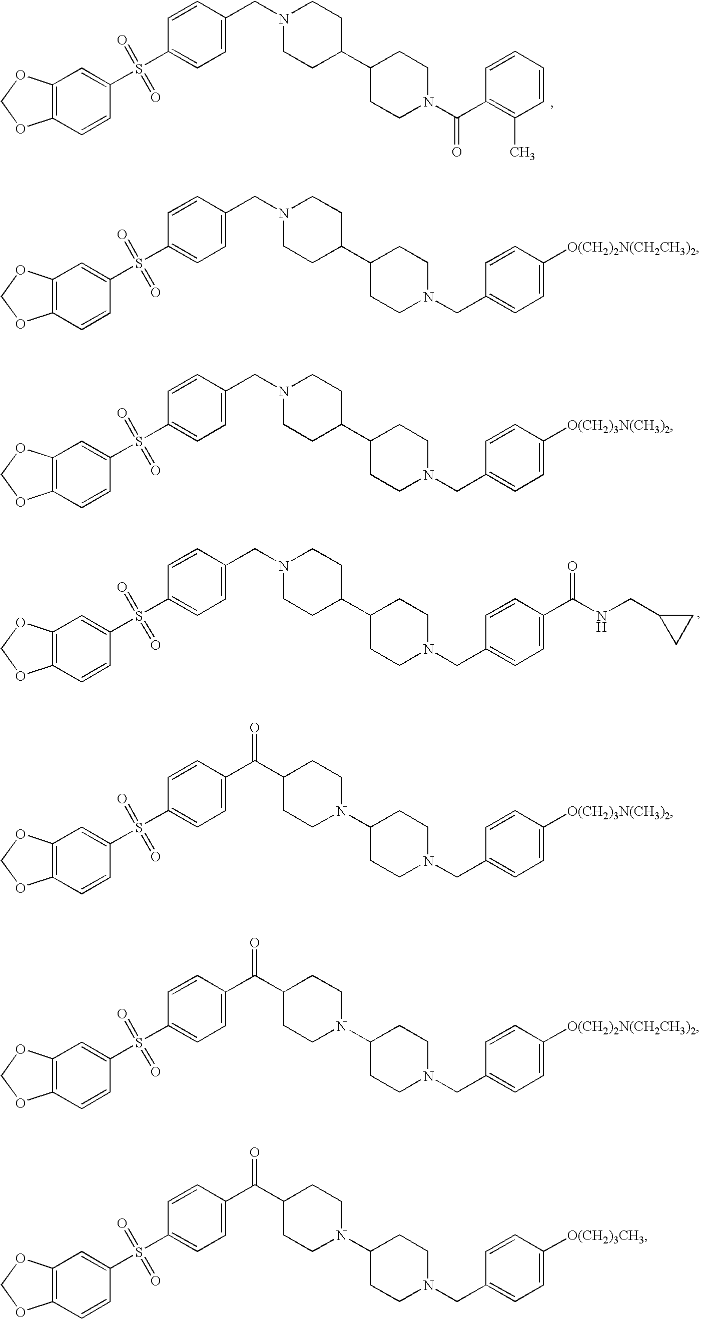

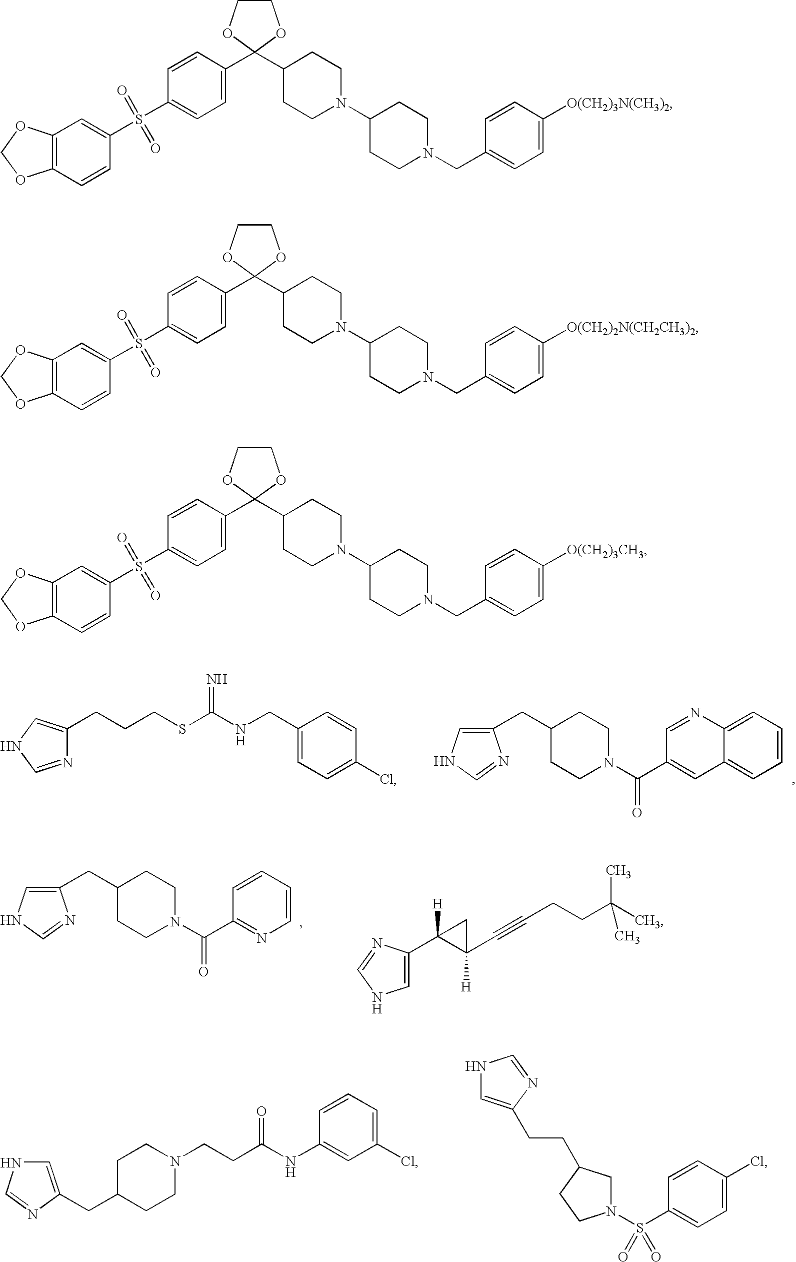

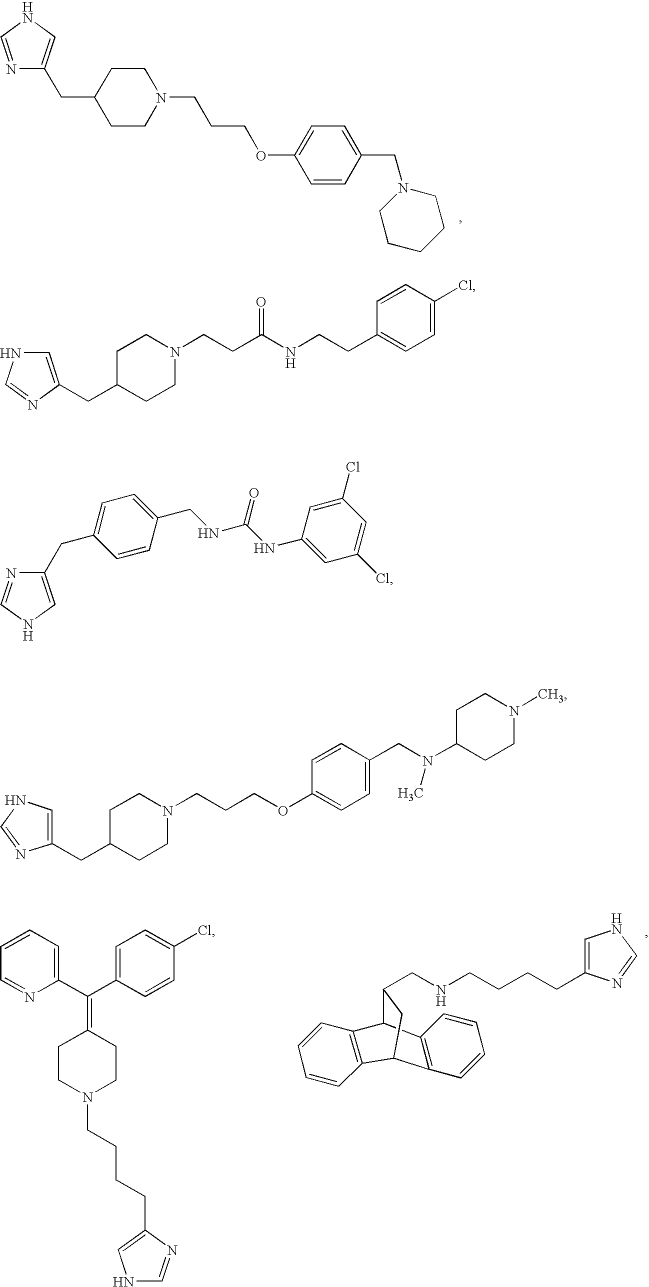

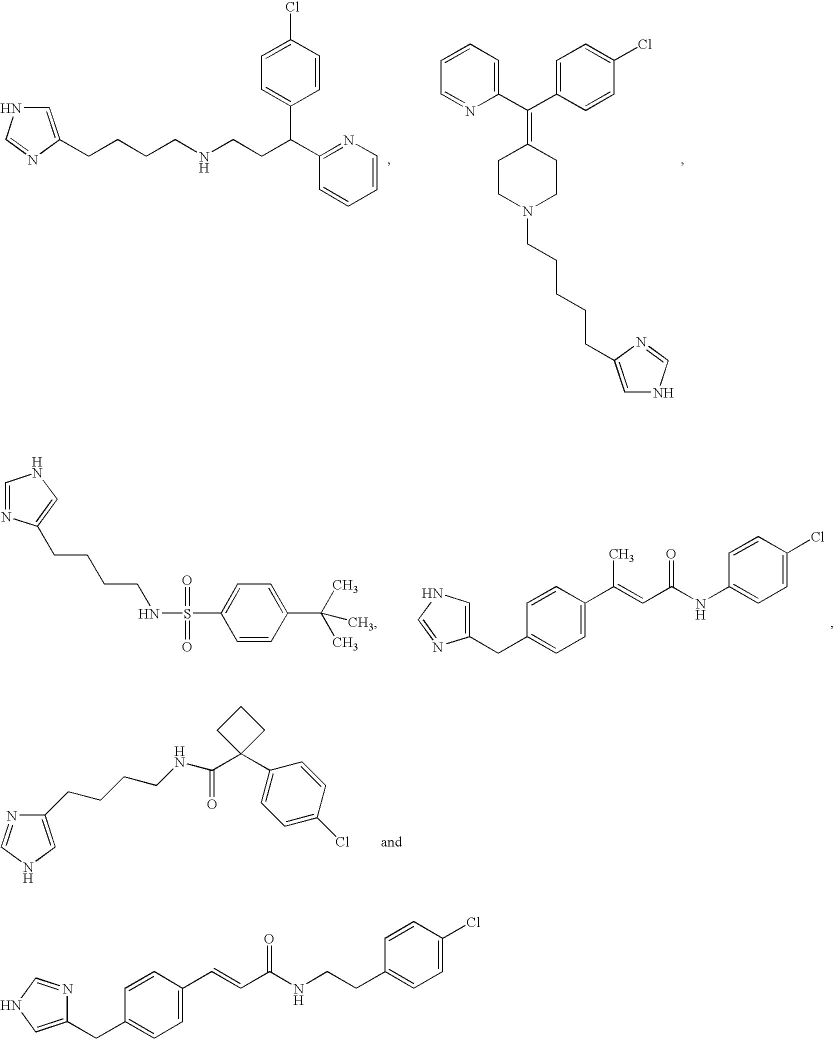

In one embodiment of the invention the combination is chlorpheniramine along with thioperamide and/or clobenpropit.

The histamine H1 receptor antagonist and the histamine H3 receptor antagonist is administered to the subject parenterally (e.g., subcutaneous, intramuscular, intraperitoneal, intravenous) or non-parenterally (e.g., topical, ocular, transdermal, sublingual, inhalation, rectal, oral).

### DETAILED DESCRIPTION OF THE INVENTION

The present invention provides methods for treating or preventing allergic skin conditions, such as urticaria, or allergic ocular conditions by administering a histamine H1 receptor antagonist and a histamine H3 receptor antagonist. Administering both an H1 and an H3 antagonist together results in a synergistically increased antihistaminic and antiallergenic effect over that of each antagonist alone. Alternatively, a single substance (e.g., a small organic molecule) which antagonizes both the histamine H1 receptor and the histamine H3 receptor (dual H1/H3 antagonist) can be administered.

The terms “subject” includes any organism, preferably a mammal (e.g., dog, cat, rat, mouse, rabbit, horse, pig and guinea pig) and most preferably a human.

The term histamine H1 receptor refers to receptors from any organism, preferably a human. An example of a human histamine H1 receptor is set forth under Genbank Accession No. AY136743. The term histamine H3 receptor refers to receptors from any organism, preferably a human. An example of a human histamine H3 receptor is set forth under Genbank Accession No. AB045369.

The terms “H1” and “H1 receptor” both refer to a histamine H1 receptor. The terms “H3” and “H3 receptor” both refer to a histamine H3 receptor.

The term “i.d.” means intradermal. The term “i.p.” means intraperitoneal. The term “i.v.” means intravenous.

### Antagonists

Histamine H3 receptor antagonists of the present invention are exemplified by the compounds shown below in Table 1.

TABLE 1
Histamine H3 receptor antagonists.

| Formula | |
|---|---|
| Number | Structure |
| 1 | 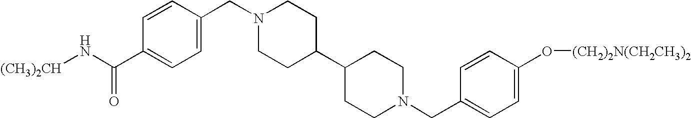 |
| 2 | 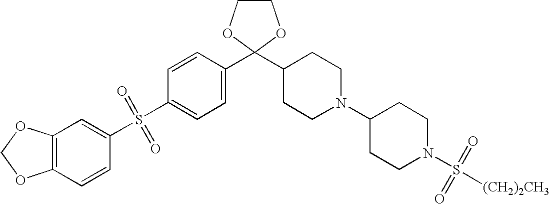 |
| 3 | 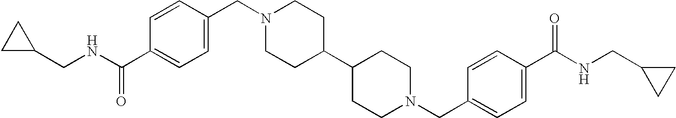 |
| 4 | 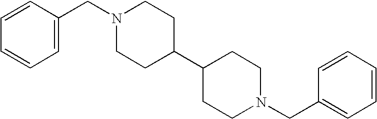 |
| 5 | 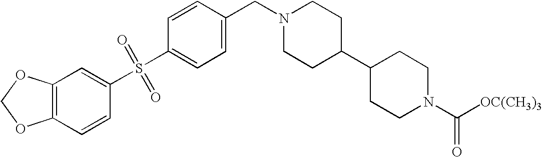 |
| 6 | 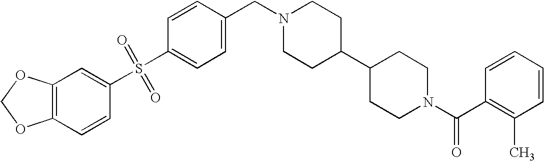 |
| 7 | 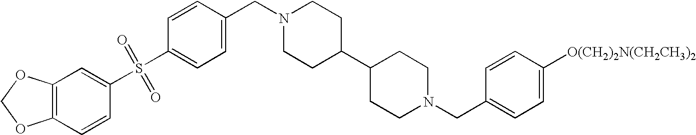 |
| 8 | 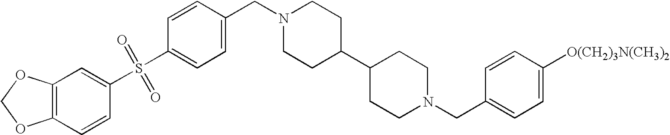 |
| 9 | 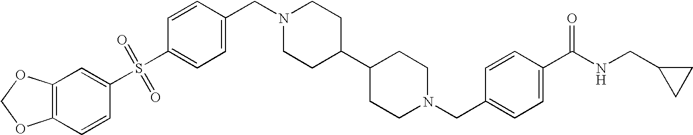 |
| 10 | 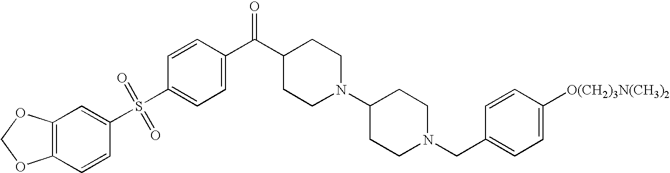 |
| 11 | 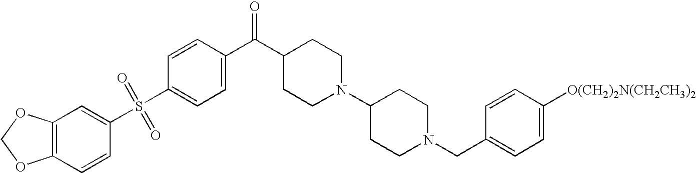 |
| 12 | 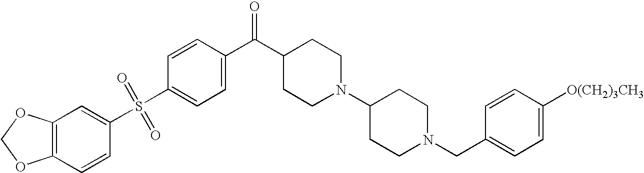 |
| 13 | 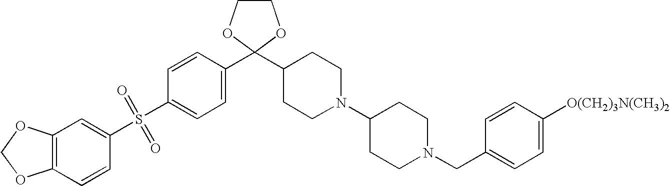 |
| 14 | 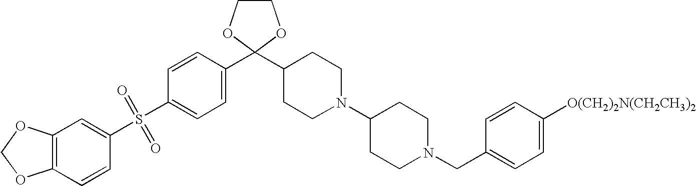 |
| 15 | 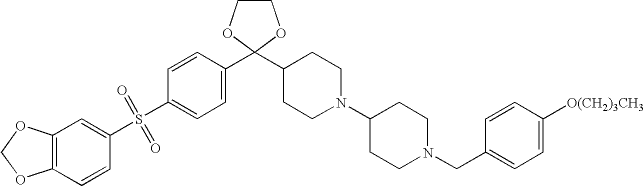 |
| 16 | 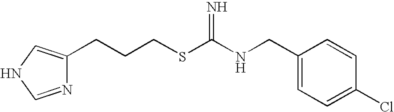 |
| 17 | 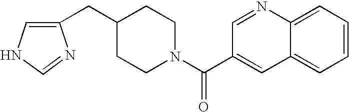 |
| 18 | 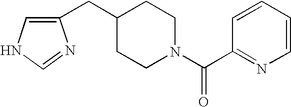 |
| 19 | 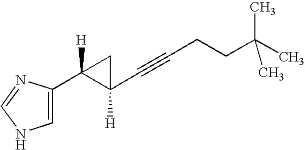 |
| 20 | 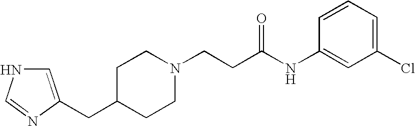 |
| 21 | 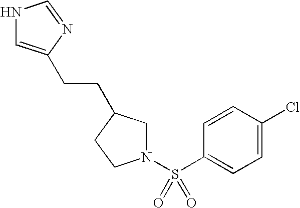 |
| 22 | 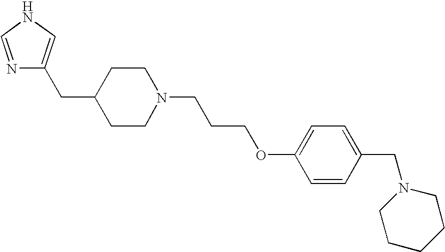 |
| 23 | 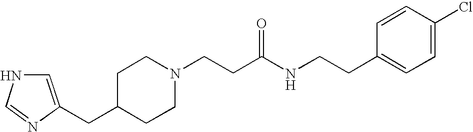 |
| 24 | 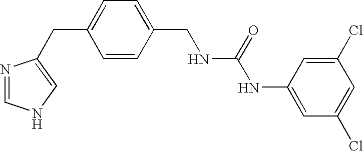 |
| 25 | 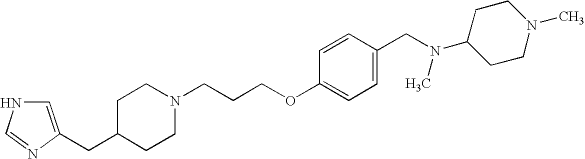 |
| 26 | 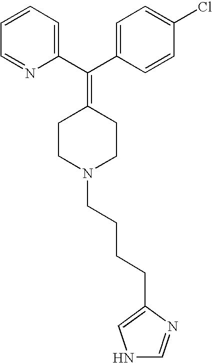 |
| 27 | 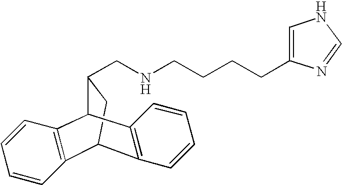 |
| 28 | 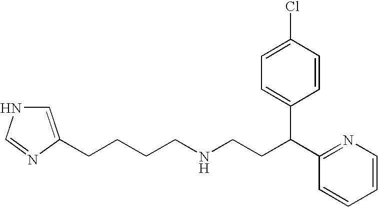 |
| 29 | 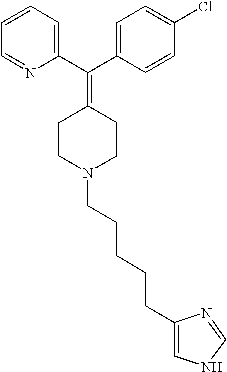 |
| 30 | 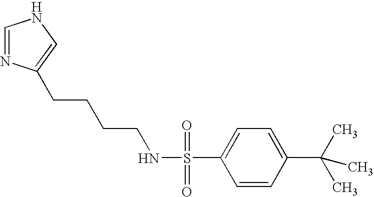 |
| 31 | 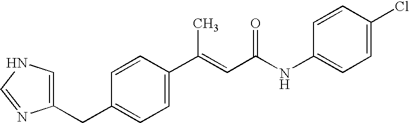 |
| 32 | 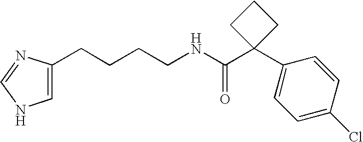 |
| 33 | 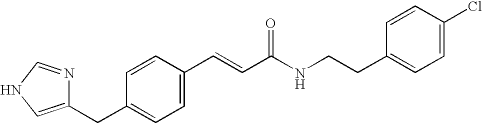 |
| 34 | 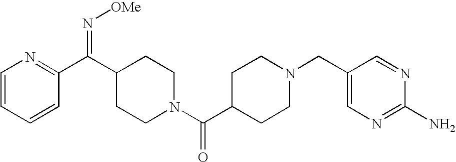 |
| 35 | 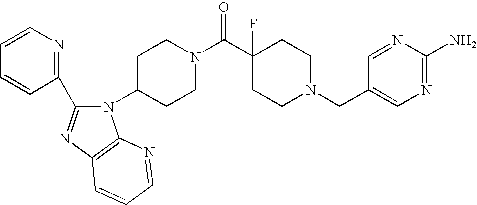 |

Other histamine H3 receptor antagonists include, without limitation: thioperamide, impromidine, burimamide, clobenpropit, impentamine, mifetidine, clozapine, S-sopromidine, R-sopromidine, ciproxifam, SKF-91486 (3-(imidazole-4-yl)-propylguanidine sulfate), GR-175737 (Clitherow, et al., (1996) Bioorg. Med. 6: 833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50), ABT331440 (PCT Publication No. WO 02/06223;

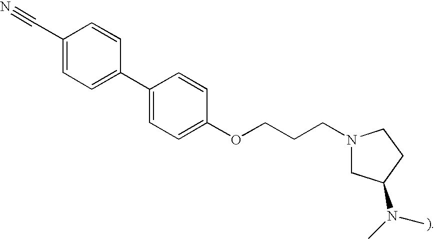

Histamine H3 receptor antagonists which are part of the present invention are disclosed in several U.S. patents, applications and publications:

PCT Publication No. WO 02/72570 discloses compounds comprising the following formula:

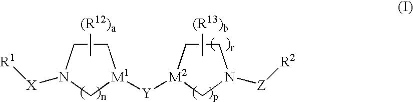

or a pharmaceutically acceptable salt or solvate thereof, wherein

(A) R¹ is selected from:

(1) aryl;

(2) heteroaryl:

(3) heterocycloalkyl

(4) alkyl.

(5)—C(O)N(R4B)₂:

(6) cycloalkyl;

(7) arylalkyl:

(8) heteroarylheteroaryl (e.g. isoxazoylthienyl or pyridythienyl): or

(9) a group selected from:

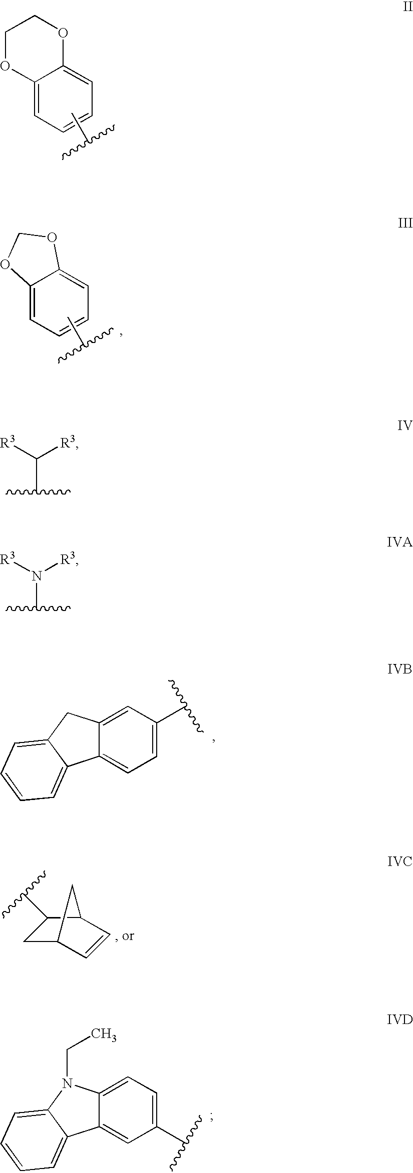

said aryl (see (A)(1) above), heteroaryl (see (A)(2) above), aryl portion of arylalkyl (see (A)(7) above), phenyl ring of formula II (see (A)(9) above), phenyl ring of formula III (see (A)(9) above), phenyl rings of formula IVB (see (A)(9) above), or phenyl rings of formula IVD (see (A)(9) above) are optionally substituted with 1 to 3 substituents independently selected from

(1) halogen (e.g. Br, F, or Cl, preferably F or Cl);

(2) hydroxyl (i.e., —OH);

(3) lower alkoxy (e.g., C₁ to C₆ alkoxy, preferably C₁ to C₄ alkoxy, more preferably C₁ to C₂ alkoxy, most preferably methoxy);

(4) —Oaryl (i.e., aryloxy);

(5) —SR²²;

(6) —CF₃;

(7) —OCF₂;

(8) —OCHF₂;

(9) —NR⁴R⁵;

(10) phenyl;

(11) NO₂,

(12) —CO₂R⁴;

(13) —CON(R⁴)₂ wherein each R⁴ is the same or different;

(14) —S(O)₂R²²;

(15) —S(O)₂N(R²⁰)₂ wherein each R²⁰ is the same or different;

(16) —N(R²⁴)S(O)₂R²²;

(17) —CN;

(18) —CH₂OH;

(19) —OCH₂CH₂OR²²;

(20) alkyl (e.g. C₁ to C₄, such as methyl);

(21) substituted phenyl wherein said phenyl has 1 to 3 substituents independently selected from alkyl, halogen, —CN, —NO₂, —OCHF₂, —Oalkyl;

(22) —Oalkylaryl (preferably —Oalkylphenyl or —Oalkyl-substituted phenyl, e.g., —OCH₂dichlorophenyl, such as —OCH₂-2,6-dichlorophenyl or —OCH₂-2-chloro-6-fluorophenyl) wherein said aryl group is optionally substituted with 1 to 3 independently selected halogens; or

(23) phenyl;

(B) X is selected from alkyl (e.g., —(CH₂)_{q}— or branched alkyl) or —S(O)₂—;

(C) Y represents

(1) a single bond (i.e., Y represents a direct bond from M¹ to M²); or

(2) Y is selected from —C(O)—, —C(S)—, —(CH₂)_{q}—, or —NR⁴C(O)—; with the provisos that:

(a) when M¹ is N, then Y is not —NR⁴C(O)—; and

(b) when Y is a bond, then M¹ and M² are both carbon;

(D) M¹ and M² are independently selected from C or N;

(E) Z is selected from: C₁-C₆ alkyl, —SO₂—, —C(O)— or —C(O)NR⁴—;

(F) R² is selected from:

(1) a six-membered heteroaryl ring having 1 or 2 heteroatoms independently selected from N or N—O (i.e., N-oxide), with the remaining ring atoms being carbon;

(2) a five-membered heteroaryl ring having 1 to 3 heteroatoms selected from nitrogen, oxygen, or sulfur with the remaining ring atoms being carbon; or

(3) an alkyl group, preferably a C₁ to C₄ alkyl group, more preferably methyl;

(4) an aryl group. e.g. phenyl or substituted phenyl (preferably phenyl), wherein said substituted phenyl is substituted with 1 to 3 substituents independently selected from: halogen, —Oalkyl, —OCF₃, —CF₃, —CN, —NO₂, —NHC(O)CH₃, or —O(CH₂)_{q}N(R^{10A})₂;

(5) —N(R^{11A})₂ wherein each R^{11A} is independently selected from: H, alkyl (e.g. i-propyl) or aryl (e.g. phenyl), preferably one R^{11A} is H and the other is phenyl or alkyl (e.g., i-propyl);

(6) a group of the formula:

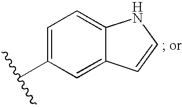

(7) a heteroarylheteroaryl group, e.g.,

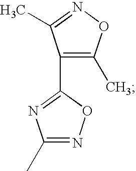

said five membered heteroaryl ring ((F)(2) above) or six-membered heteroaryl ring ((F)(1) above) is optionally substituted with 1 to 3 substituents selected from:

(a) halogen;

(b) hydroxyl;

(c) lower alkyl;

(d) lower alkoxy;

(e) —CF₃;

(f) —NR⁴R⁵;

(g) phenyl;

(h) —NO₂;

(i) —C(O)N(R⁴)₂ (wherein each R⁴ is the same or different);

—C(O)₂R⁴; or

(k) phenyl substituted with 1 to 3 substituents independently selected from: halogen, —Oalkyl, —OCF₃, —CF₃, —CN, —NO₂ or —O(CH₂)_{q}N(R^{10A})₂;

(G) R³ is is selected from:

(1) aryl;

(2) heteroaryl;

(3) heterocycloalkyl

(4) alkyl; or

(5) cycloalkyl;

wherein said aryl or heteroaryl R³ groups is optionally substituted with 1 to 3 substituents independently selected from:

(a) halogen (e.g., Br, F, or Cl, preferably F or Cl);

(b) hydroxyl (i.e., —OH);

(c) lower alkoxy (e.g., C₁ to C₆ alkoxy, preferably C₁ to C₄ alkoxy, more preferably C₁ to C₂ alkoxy, most preferably methoxy);

(d) —Oaryl (i.e., aryloxy);

(e) —SR²²;

(f) —CF₃;

(g) —OCF₃;

(h) —OCHF₂;

(i) —NR⁴R⁵;

(j) phenyl;

(k) —NO₂,

(l) —CO₂R⁴;

(m) —CON(R⁴)₂ wherein each R⁴ is the same or different;

(n) —S(O)₂R²²;

(o) —S(O)₂N(R²⁰)₂ wherein each R²⁰ is the same or different;

(p) —N(R²⁴)S(O)₂R²²;

(q) —CN;

(r) —CH₂OH;

(s) —OCH₂CH₂OR²²; or

(t) alkyl;

(H)R₄ is selected from:

(1) hydrogen;

(2) C₁-C₆ alkyl;

(3) cycloalkyl;

(4) cycloalkylalkyl (e.g., cyclopropyl-CH₂ or cyclohexyl-CH₂—);

(5) heterocycloalkylalky (e.g., tetrahydrofuranyl-CH₂—);

(6) bridged bicyclic cycloalkyl ring, such as, for example:

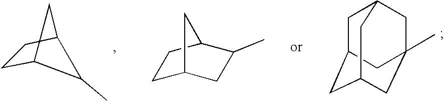

(7) aryl having a fused heterocycloalkyl ring bound to said aryl ring, preferably the heteroatoms in said heterocycloalkyl ring are two oxygen atoms, e.g. phenyl having a heterocycloalkyl ring bound to said phenyl ring, such as

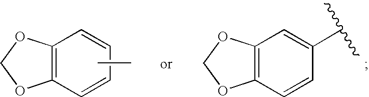

(8) aryl;

(9) arylalkyl;

(10) alkylaryl;

(11) —(CH₂)_{d}CH(R^{12A})₂ wherein d is 1 to 3 (preferably 1), and each R^{12A} is independently selected from phenyl or substituted phenyl, said substituted phenyl being substituted with 1 to 3 substituents independently selected from: halogen, —Oalkyl, —OCF₃, —CF₃, —CN, or —NO₂, e.g.,

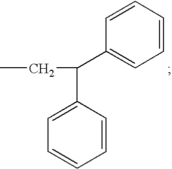

(12) heterocycloalkylheteroaryl, e.g.,

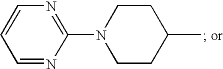

(13)—(C₁ to C₆)alkylene-O—R²² (e.g., —C₃H₆OCH₃);

wherein the aryl R⁴ group, the aryl portion of the arylalkyl R⁴ group, or the aryl portion of the alkylaryl R⁴ group is optionally substituted with 1 to 3 substituents independently selected from:

(a) halogen;

(b) hydroxyl;

(c) lower alkyl;

(d) lower alkoxy;

(e) —CF₃;

(f) —N(R²⁰)(R²⁴),

(g) phenyl;

(h) —NO₂;

(i) —C(O)N(R²⁰) (wherein each R²⁰ is the same or different),

(j) —C(O)R²²;

(i) —(CH₂)ₖ-cycloalkyl;

(j)—(CH₂)_{q}-aryl; or

(k) —(CH₂)ₘ—OR²²;

(I) each R^{4B} is independently selected from: H, heteroaryl (e.g., pyridyl), alkyl, alkenyl (e.g., allyl), a group of the formula

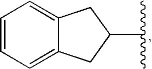

arylalkyl (e.g., benzyl), or arylalkyl wherein the aryl moiety is substitued with 1-3 substituents independently selected from: halogen (e.g. —CH₂-p-Clphenyl); preferably one R^{4B} is H;

(J) R⁵ is selected from: hydrogen, C₁-C₆ alkyl, —C(O)R²⁰ (e.g., —C(O)alkyl, such as —C(O)CH₃), —C(O)₂R²⁰, —C(O)N(R²⁰)₂ (wherein each R²⁰ is the same or different);

(K) each R^{10A} is independently selected from H or C₁ to C₆ alkyl (e.g., methyl), or each R^{10A}, taken together with the nitrogen atom to which they are bound, forms a 4 to 7 membered heterocycloalkyl ring;

(L) R¹² is

(1) selected from alkyl, hydroxyl, alkoxy, or fluoro, provided that when R¹² is hydroxy or fluoro then R¹² is not bound to a carbon adjacent to a nitrogen; or

(2) R¹² forms an alkyl bridge from one ring carbon to another ring carbon, an example of such a bridged ring system is:

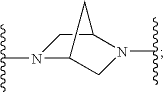

(M) R¹³ is

(1) selected from alkyl, hydroxyl, alkoxy, or fluoro, provided that when R¹³ is hydroxy or fluoro then R¹³ is not bound to a carbon adjacent to a nitrogen; or

(2) R¹³ forms an alkyl bridge from one ring carbon to another ring carbon, an example of such a bridged ring system is:

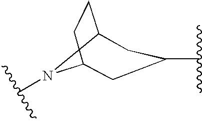

(N)R²⁰ is selected from hydrogen, alkyl, or aryl, wherein said aryl group is optionally substituted with from 1 to 3 groups independently selected from: halogen, —CF₃, —OCF₃, hydroxyl, or methoxy; or when two R²⁰ groups are present, said two R²⁰ groups taken together with the nitrogen to which they are bound form a five or six membered heterocyclic ring;

(O) R²² is selected from: heterocycloalkyl (e.g., morpholinyl or pyrrolidinyl), alkyl or aryl, wherein said aryl group is optionally substituted with 1 to 3 groups independently selected from halogen, —CF₃, —OCF₃, hydroxyl, or methoxy;

(P) R²⁴ is selected from: hydrogen, alkyl, —SO₂R²², or aryl, wherein said aryl group is optionally substituted with 1 to 3 groups independently selected from halogen, —CF₃, —OCF₃, hydroxyl, or methoxy;

(Q) a is 0 to 2;

(R) b is 0 to 2;

(S) k is 1 to 5;

(T) m is 2 to 5;

(U) n is 1, 2 or 3 with the proviso that when M¹ is N, then n is not 1;

(V) p is 1, 2 or 3 with the proviso that when M² is N, then p is not 1;

(W) q is 1 to 5; and

(X) r is 1, 2, or 3 with the proviso that when r is 2 or 3, then M² is C and p is

PCT Publication No. WO 02/32893 discloses compounds comprising the following formula:

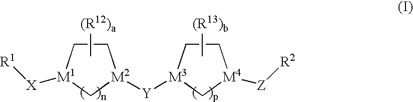

or a pharmaceutically acceptable salt or solvate thereof, wherein:

(1) R¹ is is selected from:

(a) aryl;

(b) heteroaryl;

(c) heterocycloalkyl

(d) alkyl;

(e) cycloalkyl; or

(f) alkylaryl;

wherein said R¹ groups are optionally substituted with 1 to 4 substituents independently selected from:

(1) halogen (e.g., Br, F, or Cl, preferably F or Cl);

(2) hydroxyl (i.e., —OH);

(3) lower alkoxy (e.g., C₁ to C₆ alkoxy, preferably C₁ to C₄ alkoxy, most preferably C₁ to C₂ alkoxy, more preferably methoxy);

(4) —CF₃;

(5) CF₃O—;

(6) —NR⁴R⁵;

(7) phenyl;

(8) —NO₂,

(9)—CO₂R⁴;

(10) —CON(R⁴)₂ wherein each R⁴ is the same or different;

(11) —S(O)ₘN(R²⁰) wherein each R²⁰ is the same or different H or alkyl group, preferably C₁ to C₄ alkyl, most preferably C₁-C₂ alkyl, and more preferably methyl;

(12) —CN; or

(13) alkyl; or

(2) R¹ and X taken together form a group selected from:

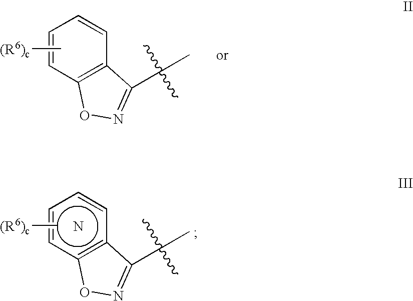

(3) X is selected from: ═C(O), ═C(NOR³), ═C(NNR⁴R⁵),

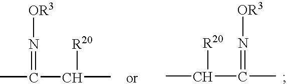

(4) M¹ is carbon;

(5) M² is selected from C or N;

(6) M³ and W are independently selected from C or N;

(7) Y is selected from: is —CH₂—, ═C(O), ═C(NOR²⁰) (wherein R²⁰ is as defined above), or ═C(S);

(8) Z is a C₁-C₆ alkyl group;

(9) R² is a five or six-membered heteroaryl ring, said six-membered heteroaryl ring comprising 1 or 2 nitrogen atoms with the remaining ring atoms being carbon, and said five-membered heteroaryl ring containing 1 or 2 heteroatoms selected from: nitrogen, oxygen, or sulfur with the remaining ring atoms being carbon; said five or six membered heteroaryl rings being optionally substituted with 1 to 3 substituents independently selected from: halogen, hydroxyl, lower alkyl, lower alkoxy, —CF₃, CF₃O, —NR⁴R⁵, phenyl, —NO₂, —CO₂R⁴, —CON(R⁴)₂ wherein each R⁴ is the same or different, —CH₂NR⁴R⁵, —(N)C(NR⁴R⁵)₂, or —CN;

(10) R³ is selected from:

(a) hydrogen;

(b) C₁-C₆ alkyl;

(c) aryl;

(d) heteroaryl;

(e) heterocycloalkyl;

(f) arylalkyl (e.g., aryl(C₁ to C₄)alkyl, e.g., —(CH₂)_{w}aryl wherein w is 1 to 4, preferably 1 or 2, and most preferably 1, such as, for example —CH₂phenyl or —CH₂substituted phenyl);

(g) —(CH₂)ₑ—C(O)N(R⁴)₂ wherein each R⁴ is the same or different,

(h) —(CH₂)ₑ—C(O)OR⁴;

(i) —(CH₂)ₑ—C(O)R³⁰ wherein R³⁰ is a heterocycloalkyl group, such as, for example, morpholinyl, piperidinyl, piperazinyl or pyrrolidinyl, including

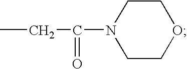

(j) —CF₃; or

(k) —CH₂CF₃;

wherein said aryl, heteroaryl, heterocycloalkyl, and the aryl portion of said arylalkyl are optionally substituted with 1 to 3 (preferably 1) substituents selected from: halogen (e.g., F or Cl), —OH, —OCF₃, —CF₃, —CN, —N(R⁴⁵)₂, —CO₂R⁴⁵, or C(O)N(R⁴⁵)₂, wherein each R⁴⁵ is independently selected from: H, alkyl, alkylaryl, or alklaryl wherein said aryl moiety is substituted with 1 to 3 substituents independently selected from —CF₃, —OH, halogen, alkyl, —NO₂, or —CN;

(11) R⁴ is selected from: hydrogen, C₁-C₆ alkyl, aryl, alkylaryl, said aryl and alkylaryl groups being optionally substituted with 1 to 3 substituents selected from: halogen, —CF₃, —OCF₃, —OH, —N(R⁴⁵)₂, —CO₂R⁴⁵, C(O)N(R⁴⁵)₂, or CN; wherein R⁴⁵ is as defined above;

(12) R⁵ is selected from: hydrogen, C₁-C₆ alkyl, —C(O)R⁴, —C(O)₂R⁴, or —C(O)N(R⁴)₂ wherein each R⁴ is independently selected, and R⁴ is as defined above;

(13) or R⁴ and R⁵ taken together with the nitrogen atom to which they are bound forms a five or six membered heterocycloalkyl ring (e.g., morpholine);

(14) R⁶ is selected from: alkyl, aryl, alkylaryl, halogen, hydroxyl, lower alkoxy, —CF₃, CF₃O—, —NR⁴R⁵, phenyl, —NO₂, CO₂R⁴, —CON(R⁴)₂ wherein each R⁴ is the same or different, or —CN;

(15) R¹² is selected from: alkyl, hydroxyl, alkoxy, or fluoro;

(16) R¹³ is selected from: alkyl, hydroxyl, alkoxy, or fluoro;

(17) a (subscript for R¹²) is 0 to 2;

(18) b (subscript for R¹³) is 0 to 2;

(19) c (subscript for R⁶) is 0 to 2;

(20) e is D to 5;

(21) m is 1 or 2;

(22) n is 1, 2 or 3; and

(23) p is 1, 2 or 3, with the proviso that when M³ and M² are both nitrogen, then p is 2 or 3 (i.e., p is not 1 when M³ and M² are both nitrogen).

U.S. Pat. No. 5,463,074 discloses compounds comprising the following formula:

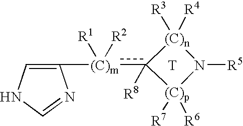

or a pharmaceutically acceptable salt or solvate, wherein:

(A) m is an integer selected from the group consisting of: 0, 1, and 2;

(B) n and p are integers and ere each independently selected from the group consisting of: 0, 1, 2, and 3 such that the sum of n and p is 2 or 3 such that when the sum of n and p is 2. T is a 4-membered ring and when the sum of n and p is 3. T is a 5-membered ring;

(C) each R¹, R², R³, R⁴, R⁶, R⁷, and R⁸ is independently selected from the group consisting of:

(1) H;

(2) C₁ to C₆ alkyl;

(3) C₃ to C₆ cycloalkyl: and

(4) —(CH₂)_{q}—R⁹ wherein q is an integer of: 1 to 7, and R⁹ is selected from the group consisting of: phenyl, substituted phenyl, —OR¹⁰, —C(O)OR¹⁰, —C(O)R¹⁰, —OC(O)R¹⁰, —C(O)NR¹⁰R¹¹, CN and —SR¹⁰ wherein R¹⁰ and R¹¹: are as defined below, and wherein the substituents on said substituted phenyl arc each independently selected from the group consisting of: —OH, C₁ to C₆)alkyl, halogen C₁ to C₆ alkyl, —CF₃, —CN, and —NO₂, and wherein said substituted phenyl contains form 1 to 3 substituents; examples of —(CH₂)₁—R⁹ include benzyl, substituted benzyl and the like, wherein the substituents on the substituted benzyl are as defined above for said substituted phenyl;

(D) R⁵ is selected from the group consisting of:

(2) C₁ to C₂₀ alkyl;

(3) C₃ to C₆ cycloalkyl;

(4) —C(O)OR¹⁰; wherein R¹⁰ is the same as R¹⁰ defined below except that R¹⁰ is not H;

(5) —C(O)R¹⁰;

(6) —C(O)NR¹⁰R¹¹;

(7) allyl:

(8) propargyl; and

(9) —(CH₂)_{q}—R⁹, wherein q and R⁹ are as defined above with the proviso that when q is 1 when R⁹ is not —OH or —SH;

(E) R¹⁰ and R¹¹ are each independently selected from the group consisting of: H, C₁ to C₆ alkyl, and C₃ to C₆ cycloalkyl; and, for the substituent —C(O)NR¹⁰R¹¹, R¹⁰ and R¹¹, together with the nitrogen to which they are bound, can form a ring having 5, 6, or 7 atoms;

(F) the dotted line ( . . . ) represents a double bond that is optionally present when m is 1, and T is a 5-membered ring, and n is not 0, and p is not 0 (i.e., the nitrogen in the ring is not bound directly to the carbon atom bearing the double bond), and when said double bond is present then R² and R⁸ are absent;

(G) when m is 2, each R¹ is the same or different substituent for each m, and each R² is the same or different substituent for each m;

(H) when n is 2 or 3, each R³ is the same or different substituent for each n, and each R⁴ is the same or different substituent for each n, and

(I) when p is 2 or 3, each R⁶ is the same or different substituent for each p, and each R⁷ is the same or different substituent for each p.

U.S. Pat. No. 5,633,250 discloses compounds comprising the following formula:

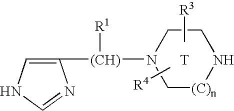

or a pharmaceutically acceptable salt or solvate thereof, wherein:

(A) n is 1 or 2, such that when n is 1 then ring T is a six membered ring, and when n is 2 the ring T is a seven membered ring;

(B) R¹ is selected from the group consisting of:

(1) H;

(2) C₁ C₆ alkyl;

(3) allyl; and

(4) propargyl;

(C) R³ and R⁴ are independently selected from the group consisting of:

(1) H;

(2) C₁ to C₆ alkyl;

(3) allyl;

(4) propargyl; and

(5) —(CH₂)_{q}—R⁵ wherein q is an integer of: 1 to 7, and R⁵ is selected from the group consisting of: phenyl substituted phenyl, —OR⁶, —C(O)OR⁶, —C(O)R⁶, —OC(O)R⁶, —C(O)NR⁶R⁷, CN and —SR⁶ wherein R⁶ and R⁷ are as defined below, and wherein the substituents on said substituted phenyl are each independently selected from the group consisting of: —OH, —O—(C₁ to C₆)alkyl, halogen, C₁ to C₆ alkyl, —CF₃, —CN, and —NO₂, and wherein said substituted phenyl contains from 1 to 3 substituents;

(D) R⁶ and R⁷ are each independently selected from the group consisting of: H and C₁ to C₆ alkyl; and

(E) R³ and R⁴ are each independently bound to the same or different carbon atom of ring T.

Those skilled in the are will appreciate that the total number of substituents on each —(Cₘ— is two, and that such substituents are independently selected from the group consisting of H, R₃, and R₄, such that there is only one R³ and one R⁴ substituent in ring T.

U.S. Pat. No. 6,034,251 discloses compounds comprising the following formula:

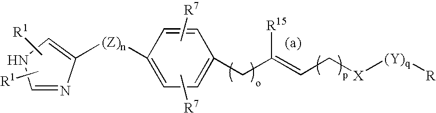

or a pharmaceutically acceptable salt or solvate thereof, wherein:

the double bond (a) is E or Z (that is the double bond to the carbon atom having the R¹⁵ substituent is of the E or Z configuration);

each R³ is independently selected from the group consisting of hydrogen, lower alkyl, trihalomethyl, phenyl and benzyl;

each R⁷ is independently selected from the group consisting of hydrogen, lower alkyl, halogen, trihalomethyl, NR¹⁰R¹¹, or a group OR¹⁰, whereby R¹⁰ and R¹¹ are independently selected from hydrogen, lower alkyl or trihalomethyl;

X is —CONR⁵—; —SO₂—, —S—; —CO—; —COO—; —CN(OR⁵)NR⁵—; —C(NR⁵)NR⁵—; —SONR⁵—; —SO₂NR⁵— and, provided p is not zero, X may also be —O—; —NR⁵—; —NR⁵CONR⁵—; —OCONR⁵—; —O—CO— or —NR⁵CO—;

Y is C₁-C₃-alkyl, optionally substituted at any carbon atom of the group by one substituent R⁵;

Z is C(R¹)₂; wherein no more than two R¹ groups are other than hydrogen;

n is 1 or 2;

m is 0 or 1;

p is 0 or 1;

q is 0 or 1;

R is selected from C₃ to C₇ cycloalkyl, heterocyclic groups, aryl or heteroaryl, wherein said R groups are optionally substituted with 1-3 substituents as defined below;

each R⁵ independently represents hydrogen, lower alkyl or poly-haloloweralkyl; and

R¹⁵ represents H or lower alkyl (e.g., methyl).

U.S. Pat. No. 6,100,279 discloses compounds comprising the following formula

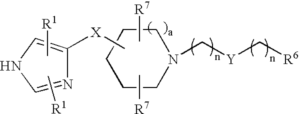

or pharmaceutically acceptable salts or solvates thereof, wherein:

X is a straight chain alkyl group having 1 to 7 carbon atoms or an alkene or alkyne group with 2 to 4 carbon atoms; wherein said alkyl or alkyne groups are optionally substituted with up to two (i.e., 1 or 2) R⁷ groups,

n is 0, 1 or 2,

m and p arc 0 to 4;

when m is 0 to 4, Y represents —SO₂—; —CS—; —CO—, —CONR⁵—; —CO(CH₂)_{w}O— (with w being 1 to 4); —COO—; —CON(OR⁵)—; —C(NR⁵) NR⁵—; —SO₂NR⁵— or —CSNR⁵—,

when m is 2 to 4, Y represents all the groups above when m is 0 to 4 and, in addition, Y represents —CHOR⁵—; —O—; —NR⁵CONR⁵—; —NR⁵CO—, —NR⁵; —OCONR⁵—; —NR⁵C(NR⁵)NR⁵—, NR⁵CSNR⁵; —NR⁵C⁵— or —NR⁵SO₂—; —NR⁵C(O)O—; or —CSNR⁵—;

each R⁵ independently represents hydrogen, alkyl or benzyl;

R⁶ represents aryl, heteroaryl, or a 3- to 7-membered heterocyclic group having one to three heteroatoms in the ring, wherein the heteroatoms are selected from N, S and O, and wherein said R⁶ group is optionally substituted by one to three substituents as defined below;

when Y is —SO₂—, then R⁶, in addition to the above groups, also represents alkyl having 1 to 7 carbon atoms or a group —NR¹⁰R¹¹ wherein R¹⁰ and R¹² are independently selected from 11 alkyl or trihalomethyl;

each R¹ is independently hydrogen, alkyl or trihalomethyl;

each R¹ is independently selected from hydrogen, alkyl, trihalomethyl, phenyl or benzyl, wherein aid phenyl and benzyl art optionally substituted by one to three substituents independently selected from of alkyl, halogen, trihalomethyl, CN, NO₂, OR¹⁰ or NR¹⁰ R¹¹, wherein R¹⁰ and R¹¹ are as above defined.

U.S. Pat. No. 5,578,616 discloses compounds comprising the following formula:

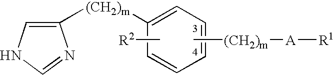

wherein:

A is selected from —O—CO—NR¹—, —CO—, —NR¹—CO—NR¹—, —NR¹—CO—, —NR¹—, —O—, —CO—NR¹—, —CO—O—, and —C(:NR¹)—NR¹—;

the groups R¹, which may be the same or different when there are two or three such groups in the molecule of formula L, are selected from hydrogen, and lower alkyl, aryl, cycloalkyl, heterocyclic and heterocyclyl-alkyl groups, and groups of the formula —CH₂)ᵥ-G, where G is selected from CO₂R³, COR³, CONR³R⁴, OR³, SR³, NR³R⁴, heteroaryl and phenyl, which phenyl is optionally substituted by halogen, lower alkoxy or polyhaloloweralkyl, and y is an integer from 1 to 3;

R² is selected from hydrogen and halogen atoms, and alkyl, alkenyl, alkynyl and trifluoromethyl groups, and groups of the formula OR³, SR³ and NR³R⁴;

R³ and R⁴ are independently selected from hydrogen, and lower alkyl and cycloalkyl groups, or R³ and R⁴ together with the intervening nitrogen atom can form a saturated ring containing 4 to 6 carbon atoms that can be substituted with one or two lower alkyl groups;

with the proviso that, when y is 1 and G is OR³, SR³ or NR³R⁴, then neither R³ nor R⁴ is hydrogen;

the group —(CH₂)ₙ-A-R¹ is at the 3- or 4-position, and the group R² is at any free position;

m is an integer from 1 to 3;

and n is 0 or an integer from 1 to 3;

U.S. Pat. No. 5,990,147 discloses compounds comprising the following formula:

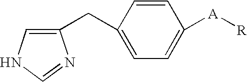

or a pharmaceutically acceptable acid addition salt or solvate thereof (or tautomer thereof, wherein:

A is —CH₂—NH—CO—NH—; —CH₂—O—CO—NH— or —CH₂CH₂—CO—NH—(CH₂)ₘ—;

m is 0, 1 or 2;

R is the group

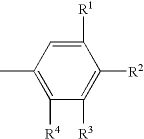

wherein at least two of R¹, R², R³ and R⁴ are hydrogen and the two others are independently selected from H, halogen (e.g. Br, I, F, or Cl), CH₃, CF₃, OCH₃, OCF₃, or CN; and

with the proviso, that when A is —CH₂—O—NH— and R¹, R³ and R⁴ are all hydrogen, then R² can not be Cl.

U.S. Pat. No. 5,807,872 discloses compounds comprising the following formula:

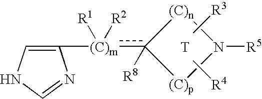

or a pharmaceutically acceptable salt or solvate thereof, wherein:

(A) m is an integer selected from the group consisting of: 1 and 2;

(B) n and p are integers and are each independently selected from the group consisting of: 0, 1, 2, 3, and 4 such that the sum of n and p is 4 and T is a 6-membered ring;

(C) R³ and R⁴ arc each independently bound to the same or different carbon atom of ring T such that there is only one R³ group and one R⁴ group in ring T, and each R¹, R², R³, and R⁴ is independently selected from the group consisting of:

(1) H;

(2) C₁ to C₆ alkyl; and

(3) —(CH₂)_{q}—R⁶ wherein q is an integer of: 1 to 7, and R⁶ is selected from the group consisting of: phenyl, substituted phenyl, —OR⁷, —C(O)OR⁷, —C(O)R⁷, —OC(O)R⁷, —C(O)NR⁷R⁸, CN and —SR⁷ wherein R⁷ and R¹⁴ are as defined below, and wherein the substituents on said substituted phenyl are each independently selected from the group consisting of: —OH, —O—(C₁ to C₆)alkyl, halogen, C₁ to C₆ alkyl, —CF₃, —CN, and —NO₂, and wherein said substituted phenyl contains from 1 to 3 substituents;

(D) R⁵ is selected from the group consisting of:

(1) 11;

(2) C₁ to C₂₀ alkyl;

(3) C₃ to C₆ cycloalkyl,

(4) —C(O)OR⁷; wherein R⁷ is the same as R⁷ defined below except that R⁷ is not H;

(5) —C(O)R⁷;

(6) —C(O)NR⁷R⁸,

(7) allyl;

(8) propargyl; and

(9) —(CH₂)_{q}—R⁶ wherein q and R⁶ are as defined above, and when q is equal to 1, then R⁶ is not OH or SH,

(E) R⁷ and R⁸ arc each independently selected from the group consisting of: 11, C₁ to C₆ alkyl, and C₃ to C₆ cycloalkyl;

(F) the dotted line (------) represents a double bond that is optionally present when m is 1, and n is not 0, and p is not 0 (i.e., the nitrogen in the ring is not bound directly to the carbon atom hearing the double bond), and when said double bond is present then R² is absent; and

(G) when m is 2, each R¹ is the same or different substituent for each m, and each R² is the same or different substituent for each m, and at least two of the substituents R¹ and or R² are H.

Those skilled in the art will appreciate that the total number of substituents on each of the —(C)ₘ— and —(C)ₚ— groups is two, and that such substituents are independently selected from the group consisting of hydrogen, R³ and R⁴, such that there is a total of only one R³ and one R⁴ substituent in ring T

The following PCT Publications disclose H3 antagonists and H1/H3 dual antagonists which may be used in the present invention: PCT Publication No. WO 02/24658 discloses compounds comprising the following formula:

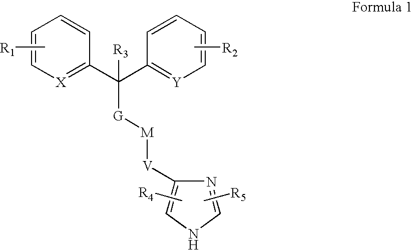

wherein

G is selected from the group consisting of C₁-C₈ alkyl or a bond;

M is a moiety selected from the group consisting of —C═C—, —C═C—, —C(═NR⁷)—NR⁶—, —NR⁶—C(═NR⁷)—, —NR⁶—C(O)—NR⁶—, —NR⁶—C(O)O—, —O—C(O)NR⁶—, —NR⁶—C(O)—, —C(O)NR⁶—, —O—, —NR⁶—, —C(O)—, —NR⁶R—, and

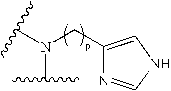

p is 1-6

V is C₁-C₆ alky;

X and Y may be the same or different and are independently selected from the group consisting of N, CH, or N-oxide, with the proviso that at least one of X and Y is N or N-oxide;

R¹ and R² may each number 1-4 and are independently selected from the group consisting of hydrogen, lower alkyl, lower alkoxy, halogen, polyhalolower alkyl, —OH, —N(R⁶)₂, —NO₂, —CN, —COOR⁶, —CONR⁶R⁸, and

—NR⁶—C(O)—R⁷ (wherein R⁷ is not —OH or —CN):

R³ is selected from hydrogen, lower alkyl, lower alkoxy, hydroxyl, polyhalolower alkyl, and a bond forming a double bond towards the moiety G when G is C₁-C₆ alkyl:

R⁴ and R⁵ are independently selected from the group consisting of hydrogen, lower alkyl, and polyhalolower alkyl;

R⁶ and R⁸ are independently selected from hydrogen, lower alkyl, aralkyl, alkylaryl, polyhalolower alkyl, substituted or unsubstituted phenyl; and substituted or unsubstituted benzyl; and

R⁷ is selected from H, OH, alkoxy, cyano, phenyl, substituted phenyl, benzyl, and substituted benzyl;

with the proviso that when G is a bond and when M is either —O— or —O—C(O)—NR⁶—, then one of X and Y is N; and with the further proviso that when R² is —OH or alkoxyl, and G is a bond, then M≠O or NR⁶.

PCT Publication No. WO 02/24659 discloses compounds comprising the following formula:

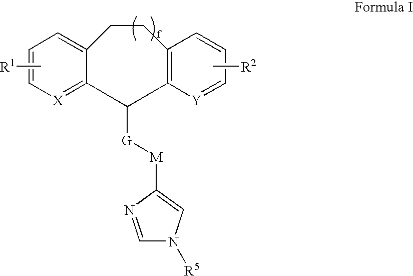

wherein:

f=0, 1 or 2;

X and Y are independently selected from the group consisting of N, CH or N-oxide;

G is a moiety selected from the group consisting of the moieties II, III and IV with the top end of said II, III and IV being linked to the tricyclic moiety and the bottom end of II, III and IV being linked to M:

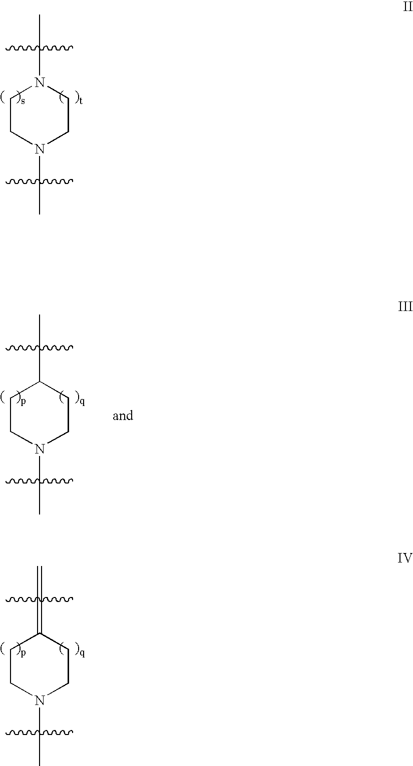

where s=t=1 or 2; and p=q=0, 1 or 2;

M is a moiety selected from the group consisting of C₁-C₈ alkyl;

—C(O)—CH₂)_{y}—; —CH₂)ₓ-A-(CH₂)_{y}—; —C(O)—O—(CH₂)_{d}—; and —C(O)NR³—(CH₂)_{d}—; where A=O, S(O)ᵣ—, and —NR⁴—;

n=0, 1, 2 or 3;

x is a whole number in the range 2-5;

y is a whole number in the range 0-5;

d is a number in the range 0-5;

r=0, 1 or 2;

R¹ and R² may each number 1-3 and are independently selected from the group consisting of hydrogen, lower alkyl, lower alkoxy, halogen, OCF₃, OCHF₂, —OH, and —N(R⁴)₂;

R³ is selected from the group consisting of hydrogen, lower alky, and polyhaloloweralkyl;

R⁴ is selected from hydrogen, lower alkyl, polyhalolower alkyl; and

R⁵ is H, C₁-C₆ alkyl or OH.

PCT Publication No. WO 02/44141 discloses compounds comprising the following formula:

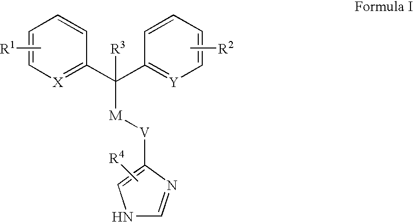

M is a moiety having a general structure shown in Formula I or III:

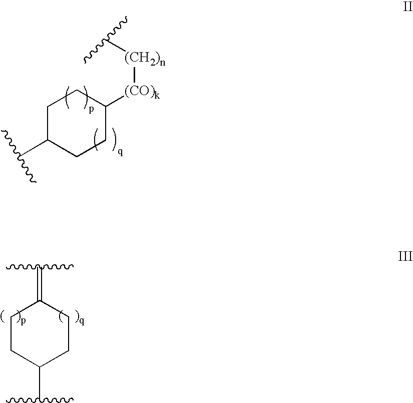

where k=0 or 1, n 0-5, and p=q=0, 1 or 2 with the proviso that when M is Formula III, R³ is absent;

V is a moiety selected from the group consisting of C₁-C₈ alkyl; —(CH₂)ₓ-A-(CH₂)_{y}—; and —(CH₂)_{c}-A-(CH₂)ₘ—C(O)N(R⁷)—(CH₂)_{d}—, where A is —O—, —S(O)ᵣ—, and —NR⁷—;

m=0, 1, 2 or 3; x is a whole number in the range 2-8; y is a whole number in the range 1-5; c is a whole number in the range 2-4; and r=0, 1 or 2; d is a number in the range 0-5;

X and Y are independently selected from the group consisting of N, CH, and N(O);

Z is selected from the group consisting of N, CH and N(O);

R¹ and R² may each number 1-4 and are independently selected from the group consisting of hydrogen, lower alkyl, lower alkoxy, halogen, polyhalolower alkyl, polyhalolower alkoxy, —OH, CN, NO₂, or COOR⁸;

R³ is selected from hydrogen, lower alkyl, lower alkoxy, hydroxyl, with the proviso that when n and k are both 0, then R³ is not —OH or alkoxy;

R⁴ is selected from the group consisting of hydrogen, lower alkyl, polyhalolower alkyl or —OH; and

R⁷ and R⁸ are independently selected from hydrogen, lower alkyl, substituted or unsubstituted phenyl; and substituted or unsubstituted benzyl.

PCT Publication No. WO 02 24657 discloses compounds comprising the following formula:

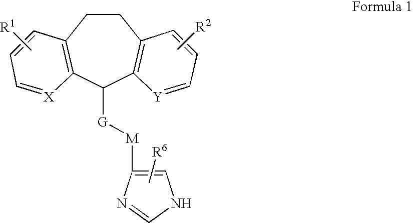

wherein

G is selected from the group consisting of —(CH₂)ᵥ—NR³—, —(CH₂)ᵥ—O—, (CH₂)ᵥ—S(O)_{z}—, —(CH₂)ᵥ—NR³—C(NR⁴)—NR³—, —(CH₂)ᵥ—O—C(O)NR³—, —(CH₂)ᵥ—NR³C(O)NR³—, —(CH₂)ᵥ—NR³C(O)O—, —(CH₂)ᵥ—NR³C(O)—, —(CH₂)ᵥC(O)NR³—;

M is a branched or unbranched alkyl group consisting of 1-6 carbon atoms, or a branched or unbranched alkenyl group consisting of 2-6 carbon atoms;

X and Y are independently selected from the group consisting of N, CH or N-oxide;

R¹ and R² may each number 1-4 and are independently selected from the group consisting of H, halogen, lower alkyl, lower alkoxy, polyhalo lower alkoxy, OH, CF₃, NH₂, NHC(O)alkyl, CN or NO₂;

R³ is independently selected from the group consisting of H, lower alkyl, substituted or unsubstituted phenyl, substituted or unsubstituted benzyl, or a group of the formula:

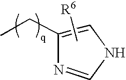

R⁴ is selected from the group consisting of H, CN, CO₂R⁵;

R⁵ is selected from the group consisting of lower alkyl and substituted or unsubstituted benzyl;

R⁶ is selected from the group consisting of H or lower alkyl;

q is 2-5;

v is 0-6; and

z is 0, 1 or 2.

Numerous chemical substances are known to have histamine H1 receptor antagonist activity. Many useful compounds can be classified as ethanolamines, ethylenediamines, alkylamines, phenothiazines or piperidines. Representative histamine H1 receptor antagonists include, without limitation: astemizole, cetirizine, azatadine, azelastine, acrivastine, brompheniramine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norasternizole, picumast, pyrilamine, promethazine, terfenadine, tripelennamine, temelastine, trimeprazine, triprolidine and

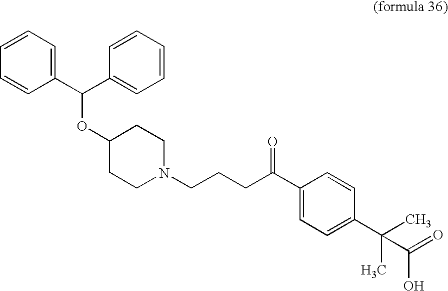

For purposes of the present invention, an “anti-histaminic” effect will be considered that symptomatic relief which has classically been considered as being obtainable by a sufferer of urticaria or an allergic ocular condition by administration of a histamine receptor antagonist including any degree of attenuation of itching, swelling and/or hive formation.

The present invention also comprises combinations comprising an H1 antagonist in association with an H3 antagonist and/or a dual H1/H3 antagonist, preferably the combination comprises one or more of the foregoing antagonists.

The term “in association” indicates that the components of the combinations of the invention can be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously at several intervals over a given period of time. Moreover, the separate components may be administered to a subject by the same or by a different route (e.g., orally, intranasally, intravenously).

### Pharmaceutical Compositions, Dosage and Administration

The present invention also includes a pharmaceutical composition comprising a histamine H1 receptor antagonist and a histamine H3 receptor antagonist and/or comprising a dual H1/H3 antagonist and a pharmaceutically acceptable carrier. The pharmaceutical compositions of the present invention may be used in the methods of the present invention. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

A pharmaceutical composition containing an H1 and an H3 antagonist and/or a dual H1/H3 antagonist can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular) and non-parenteral (e.g., oral, transdermal, intranasal, intraocular, sublingual, inhalation, rectal and topical).

Unit forms of administration include oral forms such as tablets, capsules, powders, cachets, granules and solutions or suspensions, sublingual and buccal forms of administration, aerosols, implants, subcutaneous, intramuscular, intravenous, intranasal, intraocular, subcutaneous or rectal forms of administration.

When a solid composition is prepared in the form of tablets, e.g., a wetting agent such as sodium lauryl sulfate can be added to micronized or non-micronized antagonists and mixed with a pharmaceutical vehicle such as silica, gelatin starch, lactose, magnesium stearate, talc, gum arabic or the like. The tablets can be coated with sucrose, various polymers, or other appropriate substances. Tablets can be treated so as to have a prolonged or delayed activity and so as to release a predetermined amount of active principle continuously or at predetermined intervals, e.g., by using ionic resins and the like.

A preparation in the form of gelatin capsules may be obtained, e.g., by mixing an H1 and an H3 antagonist and/or a dual H1/H3 antagonist with a diluent, such as a glycol or a glycerol ester, and incorporating the resulting mixture into soft or hard gelatin capsules.

A preparation in the form of a syrup or elixir can contain an H1 and an H3 antagonist and/or a dual H1/H3 antagonist together, e.g., with a sweetener, methylparaben and propylparaben as antiseptics, flavoring agents and an appropriate color.

Water-dispersible powders or granules can contain an H1 and an H3 antagonist and/or a dual H1/H3 antagonist mixed, e.g., with dispersants, wetting agents or suspending agents, such as polyvinylpyrrolidone, as well as with sweeteners and/or other flavoring agents.

Rectal administration may be provided by using suppositories which may be prepared, e.g., with binders melting at the rectal temperature, for example cocoa butter or polyethylene glycols.

Parenteral, intranasal or intraocular administration may be provided by using, e.g., aqueous suspensions, isotonic saline solutions or sterile and injectable solutions containing pharmacologically compatible dispersants and/or solubilizers, for example, propylene glycol or polyethylene glycol.

Thus, to prepare an aqueous solution for intravenous injection, it is possible to use a co-solvent, e.g., an alcohol such as ethanol or a glycol such as polyethylene glycol or propylene glycol, and a hydrophilic surfactant such as Tween® 80. An oily solution injectable intramuscularly can be prepared, e.g., by solubilizing the active principle with a triglyceride or a glycerol ester.

Topical administration can be provided by using, e.g., creams, ointments or gels.

Transdermal administration can be provided by using patches in the form of a multilaminate, or with a reservoir, containing an H1 and an H3 antagonist and/or a dual H1/H3 antagonist and an appropriate solvent.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an H1 and an H3 antagonist and/or a dual H1/H3 antagonist, by themselves or associated with an excipient, in powder form.

An H1 and an H3 antagonist and/or a dual H1/H3 antagonist can also be formulated as microcapsules or microspheres, e.g., liposomes, optionally with one or more carriers or additives.

Implants are among the prolonged release forms which can be used in the case of chronic treatments. They can be prepared in the form of an oily suspension or in the form of a suspension of microspheres in an isotonic medium.

The daily dose of an H1 and an H3 antagonist and/or a dual H1/H3 antagonist can be determined by a clinician and is generally dependent on the potency of the compound administered, the age, weight, condition and response of the subject.

Methods of the present invention may include administration of an H1 and an H3 antagonist and/or a dual H1/H3 antagonist along with, for example, known antihistamine, decongestant or anti-allergy agents. The administration and dosage of such agents is typically as according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art. For example, histamine antagonists of the present invention can be administered to a patient at a “therapeutically effective dosage”. A therapeutically effective dosage is any dosage which is sufficient to alleviate or prevent the symptoms or physiological effects of allergic skin and/or ocular conditions including but not limited to hay fever conjunctivitis, perennial allergic conjunctivitis, giant papillary conjunctivitis, vernal keratoconjunctivitis and atopic keratoconjunctivitis to any degree. In one embodiment of the invention, a histamine receptor antagonist of the present invention is administered to a patient or subject in need of such treatment (e.g., a patient or subject suffering from or susceptible to any of the indications mentioned herein) at a dosage of about 5 to about 2000 mg per day or about 50 mg per day to about 1900 mg/day or about 100 mg per day to about 1800 mg/day or about 300 mg per day to about 1600 mg/day or about 500 mg per day to about 1200 mg/day or about 750 mg per day to about 1000 mg/day or about 5 mg per day to about 500 mg per day or about 500 mg per day to about 1000 mg per day or about 1000 mg per day to about 2000 mg per day.

Also included in the present invention is any dosage form comprising an H1 and H3 receptor antagonist in the quantity set forth above so as to provide for convenient daily dosing of the combinations of the invention.

Typical agents which may be included along with the H1 and H3 antagonists and/or a dual H1/H3 antagonist include non-steroidal antiinflammatory drugs (NSAIDs), steroids and antiboitics (e.g., antibacterial and antifungal). NSAIDs include aspirin, acetaminophen, phenylpropionic derivatives (e.g., ibuprofen, naproxen), oxicams (e.g., piroxicam), ketorolac, celecoxib and rofecoxib. Steroids include cortisone, hydrocortisone, prednisone, prednisolone, methylprednisolone, triamcinolone, dexamethasone and betamethasone. Antibacterial agents include β-lactam antibiotics (e.g., pennicillin, amoxicillin, cloxacillin, dicloxacillin, methicillin, nafcillin, oxacillin and piperacillin), aminoglycosides (e.g., amikacin, gentamicin, kanamycin, neomycin, netilmicin, streptomycin and tobramycin), macrolides, lincomycin, and clindamycin, tetracyclines (e.g., demeclocycline, doxycycline, minocycline, oxytetracycline, tetracycline), quinolones (e.g., cinoxacin, nalidixic acid), fluoroquinolones (e.g., iprofloxacin, enoxacin, grepafloxacin, levofloxacin, lomefloxacin, norfloxacin, ofloxacin, sparfloxacin, trovafloxacin), polypeptides (e.g., bacitracin, colistin, polymyxin B), solfonamides, trimethoprim-sulfamethoxazole (TMP-SMX), chloramphenicol, vancomycin, quinupristin/dalfopristin, metronidazole, rifampin, spectinomycin and nitrofurantoin. Antifungals include amphotericin B, nystatin, itraconazole, fluconazole, ketoconazole, miconazole, sulconazole, clotrimazole, enilconazole, econazole, oxiconazole, tioconazole, terconazole, butoconazole, thiabendazole, flucytosine, griseofulvin, ciclopirox, haloprogin, naftifine, terbinafine, natamycin, tolnaftate, undecylenic acid, mafenide, dapsone, potassium iodide, silver sulfadiazine, gentian violet and carbol-fuchsin.

The H1 and H3 antagonists of the invention may be formulated together into a single composition or into two or more separate compositions for simultaneous consumption. Alternatively, for example, an H1 antagonists may be administered to a subject at a different time than when the H3 antagonist is administered; for example, each administration may be given non-simultaneously at several intervals over a given period of time.

### Indications

The methods of the present invention may be used to treat or prevent the symptoms of any medical condition or disorder which may be ameliorated by a reduction in histamine H1 receptor and histamine H3 receptor expression, activity or ligand binding.

Preferably, the methods of the present invention are used to treat or prevent the symptoms of an allergic skin condition such as urticaria. Urticaria is also known as hives, or “wheals”, which are pale red swellings of skin that generally occur in groups on any part of the skin. Each hive usually lasts a few hours before fading without a trace. New areas may develop as old areas fade. In general, hives can vary in size from as small as a pencil eraser to as large as a dinner plate and may join together to form larger swellings. Hives are usually itchy but may also burn or sting. Hives may be formed by blood plasma leakage out of small blood vessels in the skin which may be caused by the release of histamine from mast cells that lie along the blood vessels in the skin. The scope of the present invention includes methods for treatment or prevention of all types of urticaria including, but not limited to, allergic urticaria and chronic idiopathic urticaria.

The methods of the present invention may also be used to treat or prevent ocular allergic conditions such as hay fever conjunctivitis, perennial allergic conjunctivitis, giant papillary conjunctivitis, vernal keratoconjunctivitis or atopic keratoconjunctivitis; the last two types of ocular allergies listed above have potential blinding capabilities. Even the more trivial first three types of ocular allergy listed can be aggravating enough to significantly impair the quality of a patient's life.

Hay fever conjunctivitis typically occurs in individuals with sensitivities to airborne allergens such as pollens, dust, and animal danders. It is typically seasonal unlike perennial allergic conjunctivitis. Generally, seasonal allergic conjunctivitis, hay fever conjunctivitis and perennial conjunctivitis are simple allergic reactions to materials usually not producing such reactions in the normal population. The symptoms of exposure to the material to which the individual is sensitive include: itchy, running nose with sneezing, itchy, watery eyes, and ocular burning. Noticeable signs may include mild conjunctival redness, excess mucus production, and tearing.

Giant papillary conjunctivitis typically occurs in allergy-prone individuals who wear soft contact lenses. It can occur in individuals who wear other types of contact lenses, but it is more common in soft lens wearers. Typically, it occurs as a result of adherence of airborne allergens onto the surface of the contact lens, with eventual development of bumps in the conjunctiva lining the upper eyelid as the allergic/inflammatory response develops over a period of months. The symptoms of this disorder include decreased comfort with contact lens wear, mild itching, excessive contact lens movement, and excessive mucus production.

Vernal keratoconjunctivitis is an unusual, complex disorder generally involving a complex immunologic/inflammatory process. This condition has major potential for damage to the cornea and loss of vision. The condition may affect young people more often than older people and is considerably more common in males than in females. Generally, it occurs in the Spring in temperate climates and is much more common in warmer climates than in temperate or cold climates. It is particularly prevalent in the Middle East. Typically, it is characterized by the development of very large bumps on the lining of the upper eyelid and itching is a prominent symptom. Other symptoms and signs may include ocular burning, foreign body sensation, excessive tearing, excess mucus production, and blurred vision.

Atopic keratoconjunctivitis is also a serious allergic eye condition with major blinding potential. It typically occurs in young adults and adults with atopic dermatitis (eczema). Ocular itch is the primary, beginning symptom but foreign body sensation, ocular burning, excessive tearing, mucus production, and blurred vision generally, eventually occur.

### Kits

The present invention also provides kits comprising the components of the combinations of the invention in kit form. A kit of the present invention includes one or more components including, but not limited to, one or more histamine H1 antagonists, for example, as discussed herein, in association with one or more histamine H3 receptor antagonists, for example, as discussed herein. The antagonists can be formulated as a pure composition or in combination with a pharmaceutically acceptable carrier, in a pharmaceutical composition.

In one embodiment, a kit includes one or more histamine H1 antagonists, or a pharmaceutical composition thereof, in one container (e.g., in a sterile glass or plastic vial) and one or more histamine H3 antagonists, or a pharmaceutical composition thereof, in another container (e.g., in a sterile glass or plastic vial).

In another embodiment of the invention, the kit comprises a combination of the invention, including one or more histamine H1 antagonists along with one or more histamine H3 antagonists formulated together, optionally, along with a pharmaceutically acceptable carrier, in a pharmaceutical composition, in a single, common container.

If the kit includes a pharmaceutical composition for parenteral administration to a subject, the kit can include a device for performing such administration. For example, the kit can include one or more hypodermic needles or other injection devices.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The present examples are provided for further description and should not be construed to limit the present invention.

### Example 1

### Effect of Combined Histamine H1 and H3 Receptor Blockage on Cutaneous Microvascular Permeability Elicited By Compound 48/80.

In this example, the vascular effects of endogenous mast cell histamine on H3 receptors in the skin is studied. In addition, the pharmacological effect of combined blockade of H1 and H3 receptors on cutaneous microvascular permeability produced by compound 48/80 injections was evaluated.

Suppression of wheal and flare skin responses elicited by histamine (Simons, et al., (1997) Ann. Allergy Asthma Immunol. 79:530-532) or by mast cell mediator releasing agents, such as compound 48/80 (Marks, et al., (1977) Br. J. Clin. Pharmacol. 4:364-369; Goldberg, et al., Ann. Allergy 64: 179-181; Smith, et al., (1992) Lancet 339: 91-93) are well established approaches used to evaluate the peripheral actions of antiallergy drugs in preclinical and clinical studies. In this example, extravasation of Evans blue dye as a surrogate of wheal and flare responses produced by i.d. compound 48/80 was used. It was found that, given together, an H1 and H3 antagonist attenuated skin responses produced by compound 48/80 to a greater extent than either an H1 or an H3 antagonist alone in an experimentally-induced urticaria model in guinea pigs.

### Materials and Methods

Animal Care and Use. The studies were performed in accordance to the NIH GUIDE TO THE CARE AND USE OF LABORATORY ANIMALS and the Animal Welfare Act in an AAALAC-accredited program.

Evaluation of intradermal Compound 48/80 on Cutaneous Permeability. Overnight fasted adult male Hartley guinea pigs (400-500 g, Charles River, Bloomington, Mass., USA) were anesthetized with pentobarbital (35 mg/kg, i.p.). The left jugular vein was cannulated for administration of drugs. A cannula was also placed in the trachea and guinea pigs were mechanically ventilated (volume=4 ml; rate 45 breaths/min) with a small animal respirator. Intradermal (i.d.) injections of compound 48/80 (0.0001-0.01% 50 μl per injection) a histamine mast cell liberator (Lagunoff, et al., (1983) Annu. Rev. Pharmacol. Toxicol. 23: 331-351), were made along the shaven back of the guinea pig. Evans blue dye (30 mg/kg, i.v.) was given 5 minutes before compound 48/80. Test drugs were given 10 minutes before compound 48/80. At the end of the experiment, 15 minutes after compound 48/80, the guinea pigs were perfused with 200 ml of saline via the left cardiac ventricle. The tissue containing the Evans blue was removed. Dissected tissues were incubated in 1 ml formamide at 37° C. for 18 hours. To quantify the amount of dye in each sample, calorimetric measurements were performed using a SLT Lab Instruments SLT-340 AATC plate reader (Grodig, Salzburg). Tissue Evans blue concentrations were quantified by interpolation on a standard curve of dye concentrations in the range of 0.3 to 30 gg/ml.

Evaluation of Histamine H1 and H3 Blockade on Cutaneous Permeability Responses due to Intradermal Compound 48/80. The effect of dual histamine H1 and H3 receptor blockade on cutaneous microvascular permeability to graded doses of compound 48/80 (0.0001-0.01%, 50 pI, i.d.) were studied. The action of the H1 antagonist, chlorpheniramine (CTM; 1.0 mg/kg, i.v.) and the H3 antagonist, thioperamide (THIO; 1 mg/kg, i.v.), administered alone and in combination was evaluated. We also studied the pharmacology of dual histamine H1 and H3 receptor blockade with a second histamine H3 antagonist, clobenpropit (CLOB), which is structurally different from THIO. In these studies, the effect of combined treatment with CTM (1.0 mg/kg, i.v.) and CLOB (1.0 mg/kg, i.v.) were evaluated. The single high dose of CTM (1.0 mg/kg, i.v.) used in the current study was 4 times the antihistamine oral ED₅₀ activity in the guinea pig (Tozzi, et al., (1974) Agents and Action 4: 264-270). The dose of the H3 antagonists (THIO and CLOB) used were chosen based on literature values shown to be pharmacologically active in vivo (McLeod, et al., (1996) Gen. Pharmacol. 27: 1001-1007; McLeod, et al., Amer. J. Rhinol. 13: 391-399).

Drugs. Compound 48/80 and Evans blue were purchased from Sigma Chemical Co. (St. Louis Mo, USA). Thioperamide maleate and clobenpropit dihydrobromide were purchased from Research Biochemicals International (Natick, Mass., USA). Chlorpheniramine maleate was synthesized by Schering-Plough Research Institute. Drugs were dissolved in physiological saline (0.9%) and drug doses refer to their respective free bases. Control animals were given saline.

Statistics. The data are expressed as the percent inhibition of compound 48/80 response. Values shown represent the mean±SEM determined of 28-42 animals per group. Statistical significance was evaluated by a Kruskal-Wallis Test in conjunction with a Mann Whitney-U Test. Statistical significance was set at p<0.05.

### Results

Saline (50 μl) produced extravasation of Evans blue (6±1 ng/mg tissue) in the skin after i.d. administration (Table 2). Table 2 also shows that compound 48/80 (0.0001-0.01%, 50 μl), 15 minutes after i.d. administration, produced a dose-dependent increase in skin Evans blue concentration compared to saline treatment. Significant increases in Evans blue concentrations were observed at doses of compound 48/80 greater than 0.0001% (i.e., 0.0003%, 0.001%, 0.003% and 0.01%). The maximum dye leakage effect produced by compound 48/80 (0.01%) was 24±3 ng/mg tissue. Neither CTM (1.0 mg/kg, i.v.), THIO (1.0 mg/kg, i.v.) nor CLOB (0.3 mg/kg, i.v.) had any effect on baseline Evans blue leakage (i.e., dye leakage due to i.d. saline administration). Intravenous administration of CTM (1.0 mg/kg) alone attenuated the dermal effects of compound 48/80 (0.0003%, 0.001%, 0.003% and 0.01%) by 17%±4%, 31%±4%, 32%±4% and 37%±4%, respectively (Table 3). Dual treatment with the H1 antagonist, CTM (1.0 mg/kg, i.v.), and the H3 antagonist, THIO (1.0 mg/kg, i.v.), inhibited compound 48/80 (0.0003%, 0.001%, 0.003% and 0.01%) skin responses by 36%±4%, 45%±4%, 49%±4% and 54%±4%, respectively. These effects were significantly different from animals treated with CTM alone. Table 3 also shows that treatment with a combination of the H1 antagonist, CTM (1.0 mg/kg, i.v.), and the H3 antagonist, CLOB (0.3 mg/kg, i.v.), produced a similar 48/80-induced cutaneous permeability inhibitory profile to that of the CTM and THIO combination. THIO (1.0 mg/kg, i.v.) or CLOB (0.3 mg/kg, i.v.) alone had no observable effect on cutaneous permeability (Table 4).

TABLE 2
Concentration of Evans Blue Dye in the Skin after
Intradermal Injections of Compound 48/80.

| % Concentration | |
|---|---|
| of | Amount of Tissue Evans Blue |
| Compound 48/80 | (ng/mg tissue)¹ |
| 0 | 6.3 ± 1.0 |
| 0.0001 | 10.4 ± 1.2 |
| 0.0003 | 10.8 ± 1.06* |
| 0.001 | 15.1 ± 1.9* |
| 0.003 | 22.0 ± 2.3* |
| 0.01 | 24.7 ± 3.4* |

¹Each value represents the mean ± SEM. (*p < 0.05 compared to control animals).

TABLE 3
Effect of Histamine H₁ and H₃ Receptor Blockade on Cutaneous
Microvascular Permeability Responses Produced by Intradermal
Injections of Compound 48/80.

| | % Inhibition of Compound 48/80 | | | |
|---|---|---|---|---|
| | Skin Responses¹ | | | |
| Treatment | cpd 48/80 | cpd 48/80 | cpd 48/80 | cpd 48/80 |
| (mg/kg) | (0.0003%) | (0.001%) | (0.003%) | (0.01%) |
| CTM (1) | 17.3 ± 3.6 | 31.5 ± 3.7* | 31.8 ± 3.8* | 36.7 ± 3.8* |
| CTM (1) + | 35.7 ± 3.6** | 44.9 ± 4.0** | 48.8 ± 4.2** | 54.1 ± 3.5** |
| THIO (1) | | | | |
| CTM (1) + | 30.4 ± 4.2** | 48.6 ± 3.2** | 47.6 ± 3.5** | 50.9 ± 3.0** |
| CLOB (1) | | | | |

¹Each value represents the mean ± SEM (* p < 0.05 compared to vehicle;
**p < 0.05 compared to CTM).

TABLE 4
Effect of Histamine H3 Receptor Blockade on Compound
48/80-Induced Cutaneous Microvascular Permeability.

| | % Inhibition of Compound | | | |
|---|---|---|---|---|
| | 48/80-Induced Skin Response | | | |
| | 48/80 | 48/80 | 48/80 | 48/80 |
| Treatment | (0.0003%) | (0.001%) | (0.003%) | (0.01%) |
| Thioperamide (1 mg/kg) | 1 ± 7 | 26 ± 9 | 4 ± 9 | 11 ± 11 |
| Clobenpropit (0.3 mg/kg) | 1 ± 6 | 15 ± 5 | 8 ± 10 | 7 ± 8 |

### Discussion

Histamine H3 receptors were first identified by Arrang, et al., (Nature (1983) 302: 832-837) in the central nervous system where they are located, presynaptically, on histaminergic nerve terminals. Subsequently, H3 receptors have also been shown to be located on noradrenergic, cholinergic and serotonergic neurons where their activation inhibits neurotransmitter release (Arrang, et al., (1983) Nature 302: 832-837; Schlicker, et al., (1988) Naunyn-Schmiedeberg's Arch. Pharmacol.337:588-590; Schlicker, et al., (1989) Naunyn-Schmiedeberg's Arch. Pharmacol. 340: 633-638). In the periphery, specifically in the cardiovascular system, activation of histamine H3 receptors is believed to modulate sympathetic vascular responses via a prejunctional mechanism (McLeod, et al., (1996) Gen. Pharmacol. 27: 1001-1007; Malinowska, et al., (1991) Eur. J. Pharmacol. 205: 307-310; Hey, et al., (1992) Br. J. Pharmacol. 107:347-350; McLeod, et al., (1993) Br. J. Pharmacol. 110:553-558). Rizzo, et al., (Eur. J. Pharmacol. (1995) 294: 329-335) found that activation of histamine H3 receptors inhibited electrical field-induced contractions of guinea pig pulmonary arteries by attenuating noradrenaline release from sympathetic noradrenergic nerves. More recently, Valentine, et al., (Eur. J. Pharmacol. (1999) 366: 73-78) demonstrated that stimulation of H3 receptors blocks electrically-evoked contractions in human saphenous vein. Taken together, these studies may suggest that histamine H3 receptors, present on sympathetic nerves innervating arteries, may act to modulate local blood flow in skin.

The physiological role of histamine H3 receptors in the skin has not been fully elucidated. Kavanagh, et al., (Br. J. Dermatol. (1998) 138: 622-626) showed that i.d. Rα-methylhistamine, an H3 agonist, produces wheal and flare responses in human skin. Flare responses to Rx-methylhistamine were significantly attenuated by the H1 antagonist, terfenadine but not by thioperamide. Moreover, they found that thioperamide did not alter the cutaneous vascular effects of i.d. codeine phosphate, substance P and histamine. These investigators concluded that H3 receptors do not appear to inhibit histamine release in human skin. Consistent with the observations of Kavanagh, et al., (Br. J. Dermatol. (1998) 138: 622-626), we presently show that thioperamide or clobenpropit, given alone at doses that block H3 receptors in vivo, does not alter the cutaneous microvascular permeability responses elicited by compound 48/80 (McLeod, et al., (1996) Gen. Pharmacol. 27: 1001-1007; McLeod, et al., (1999) Amer. J. Rhinol. 13: 391-399). However, when histamine H1 receptors were blocked with chlorpheniramine, we demonstrated a significant H3 component contributing to the extravasation of Evans blue evoked by compound 48/80.

Without being bound by a single theory, histamine released from mast cells may activate histamine H1 and H3 receptors to produce local skin vasodilation and extravasation. Histamine H2 receptors also play a role in histamine-induced skin reactions; however, we did not evaluate H2 activity in our study (Marks, et al., (1977) Br. J. Clin. Pharmacol. 4:364-369; Miller, et al., (1989) J. Allergy Clin. Immunol. 84: 895-899). Again, without being bound by a single theory, based on several previous studies demonstrating a sympathetic noradrenergic modulatory role of H3 receptors in the cardiovascular system, mast cell histamine released by compound 48/80 may activate prejunctional H3 located on sympathetic nerves terminals to decrease vascular tone and increase blood flow in the skin (McLeod, et al., (1996) Gen. Pharmacol. 27: 1001-1007; Malinowska, et al., (1991) Eur. J. Pharmacol. 205: 307-310; Rizzo, et al., (1995) Eur. J. Pharmacol. 294: 329-335; Valentine, et al., (1999) Eur. J. Pharmacol. 366: 73-78). Consistent with this model are the results by Bolser et al., (Prog. Respir. Res. Basel, Karger, (2001) 31: 133-136.) demonstrating that H3 receptor activation inhibited the decrease in nasal blood flow and blocked the decrease in nasal airway resistance evoked by electrical stimulation of the cervical sympathetic trunk in cats. Moreover, Laurikainen, et al., (Eur. Arch. Otorhinolaryngol. (1998) 255: 119-123) recently found that H3 receptors modulate cochlear blood flow in the guinea pig. Presently, we used the extravasation of Evans blue as a surrogate for wheal and flare responses. Without being bound by a single theory, H3 receptors may contribute to the vasodilation underlying skin wheal and flare responses caused by endogenous histamine release from mast cells. The H3 mediated vasodilation may occur at the level of local pre-capillary or post-capillary blood vessels and may be the result of an attenuation of noradrenaline release from sympathetic nerves (Rizzo, et al., (1995) Eur. J. Pharmacol. 294: 329-335).

In summary, our results indicate that combined treatment with a H1 antagonist and an H3 antagonist or administration of a drug with both an H1 antagonist and an H3 antagonist is effective for the treatment of allergic skin conditions. Without being bound by a single theory, dual blockade of H1 and H3 histamine receptors may inhibit cutaneous microvascular permeability and wheal and flare responses, produced by mast cell histamine, to a greater extent that either an H1 antagonist or an H3 antagonist alone.

### Example 2

### Screening Methods for Identifying Histamine H3 Receptor Antagonists

Compounds can readily be evaluated to determine activity at histamine H3 receptors by known methods, including the guinea pig brain membrane assay and the guinea pig neurogenic ileum contraction assay, both of which are described in U.S. Pat. No. 5,352,707. Another useful assay utilizes rat brain membranes and is described by West, et al., (1990) Molecular Pharmacology 38: 610-613.

A particularly useful screening assay measures binding to sites in guinea pig brain membranes. This test is described in detail by Korte, et al., (1990) Biochem. Biophys. Res. Comm. 168: 979-986, and quantifies the displacement of bound radiolabeled N′-methylhistamine from tissues by candidate compounds. Results are expressed as “Kᵢ” values, in nanoMolar (nM) units, which values can be considered as being dissociation constants for the H3 antagonist on the H3 receptor system, or an index of antagonist affinity for the receptor.

Affinity values (Kᵢ) may determined using the following formula:

Kᵢ=IC₅₀/(1+(concentration of ligand/affinity (K_{D}) of radioligand))

The method of Korte, et al (supra) was used to analyze thioperamide and clobenpropit. The results are set forth below (see also WO 98/06394):

| Compound | Kᵢ (nM) |
|---|---|
| Thioperamide | 12 |
| Clobenpropit | 0.1 |

### Example 3

### Screening Methods for Identifying Histamine H1 Receptor Antagonists

Candidate histamine H1 receptor antagonists may be evaluated, for example, by adapting the methods set forth, above, in Example 2. Making such an adaptation would be easily done by one of ordinary skill in the art. Alternatively, there are several methods known in the art for assaying histamine H 1 receptor antagonists (Moguilevsky, et al., (1994) European Journal of Biochemistry 224: 489-495). See also Arunlakshana, et al., (1959) Br. J. Pharm. Chemoth. 14: 153-161; Ash, et al., (1966) Br. J. Pharm. Chemoth 27:427-439; Hill (1990) Pharm. Rev. 42(1): 45-83; Hill, et al., (1981) Mol. Pharm. 19: 379-387 and Trzeciakowski (1987) J. Pharm. Exp. Ther. 243: 874-880.

### Example 4

### Screening Assay for Histamine H1 and H3 Receptor Antagonists

In the present example, the affinities of several compounds for the H1 and H3 receptors was determined by a membrane binding assay.

Materials. Rat and guinea-pig brains were obtained frozen from Rockland Immunochemicals (Gilbertsville, Pa.). Cell lines expressing recombinant human receptors were generated by using standard transfection techniques. The following radioligands were obtained from Dupont NEN (Boston, Mass.): [³H]-pyrilamine, 23 Ci/mmol for H 1 binding and [³H]-N′-methylhistamine, 82 Ci/mmol for H3 binding.

Methods. Recombinant cell lines (i.e., human H1—CHO cells and human H3-HEK293) were cultured in Dulbecco's modified Eagle's medium/10% fetal bovine serum supplemented with 2 mM glutamine, penicillin (100 U/ml), and streptomycin (100 μg/ml) in a humidified 5% CO₂ atmosphere at 37° C. Selection was maintained with 0.5 mg geneticin/ml. Cells were harvested for membrane preparation by aspirating media, replacing it with Hanks' balanced salt solution/5 mM EDTA, and incubating flasks for 10 minutes at 37° C. Cells were pelleted by centrifugation at 1000×g for ten minutes at 4° C.

Membrane preparation. Membranes were prepared by disrupting cells or tissue in at least ten volumes of ice-cold 50 mM Tris-HCl, pH 7.5 at 25° C., with a Polytron. Homogenates were centrifuged ten minutes at 1000×g and the supernatants were then centrifuged for ten minutes at 50,000×g. Pellets from this centrifugation step were resuspended with a Polytron, a sample was taken for protein determination (BCA; Pierce; Rockford, Ill.), and the resuspension was again centrifuged at 50,000×g. Brain membranes were stored as pellets, cell membranes as suspensions of 1 mg protein/ml Tris buffer at −20° C.

Binding assays. Membrane (300 μg of brain membrane protein, 5-10 μg of recombinant cell membrane) was incubated with radioligand at a concentration near its KD value without or with inhibitor compounds in a total volume of 200 μl Tris buffer. Nonspecific binding was determined in the presence of 10⁻⁶ M chlorpheniramine for H1 binding or 10⁻⁶ M clobenpropit for H3 binding. Assay mixtures were incubated for 30 minutes at 30° C. in polypropylene, 96-well, deep-well plates then filtered through 0.3% polyethylenimine-soaked GF/B filters. These were washed three times with 1.2 ml of Tris buffer, dried in a microwave oven, impregnated with Meltilex wax scintillant and counted at 40% efficiency in a Betaplate scintillation counter (Wallac). IC₅₀ values were determined by interpolation or by nonlinear, least-squares, curve-fitting with the Prism program (GraphPad Software). Kᵢ values were determined in the manner of Cheng and prusoff (Cheng, et al., (1973) Biochem. Pharm. 22:3099-3108).

The data generated in these experiments are shown, below, in Table 5.

TABLE 5
Equilibrium Dissociation Constants for the Compounds
of Formulas 16-33 and 36 at Histamine Receptors H1 and H3.

| Formula | H3 Ki (nM) | H1 Ki (nM) |
|---|---|---|
| 16 | 0.06 | |
| 17 | 55 | 0% |
| 18 | 2 | |
| 19 | 3 | |
| 20 | 0.8 | 330 |
| 21 | 6 | 660 |
| 22 | 3 | 29 |
| 23 | 0.7 | |
| 24 | 15 | 0% |
| 25 | 4 | |
| 26 | 3 | 2 |
| 27 | 19 | |
| 28 | 10 | 310 |
| 29 | 8 | 5 |
| 30 | 17 | 32% |
| 31 | 470 | |
| 32 | 19 | 48% |
| 33 | 7 | 0% |
| 36 | 1% | 15 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A method for treating or preventing an allergic skin condition or an allergic ocular condition in a subject, comprising administering one or more histamine H1 receptor antagonists and one or more histamine H3 receptor antagonists to the subject.

2. The method of claim 1 wherein a histamine H1 receptor antagonist and a histamine H3 receptor antagonist are administered along with a pharmaceutically acceptable carrier in a pharmaceutical composition.

3. The method of claim 1, wherein a histamine H1 receptor antagonist and a histamine H3 receptor antagonist are present in a single dosage form.

4. The method of claim 1, wherein a histamine H1 receptor antagonist and a histamine H3 receptor antagonist are administered in separate dosage forms.

5. The method of claim 1, wherein a histamine H1 receptor antagonist is one or more members selected from the group consisting of astemizole, azatadine, azelastine, acrivastine, brompheniramine, chlorpheniramine, clemastine, cyclizine, carebastine, cyproheptadine, carbinoxamine, desloratadine, doxylamine, diphenhydramine, cetirizine, dimenhydrinate, dimethindene, ebastine, epinastine, efletirizine, fexofenadine, hydroxyzine, ketotifen, loratadine, levocabastine, mizolastine, mequitazine, mianserin, norebastine, meclizine, norastemizole, picumast, pyrilamine, promethazine, terfenadine, tripelennamine, temelastine, trimeprazine, triprolidine and

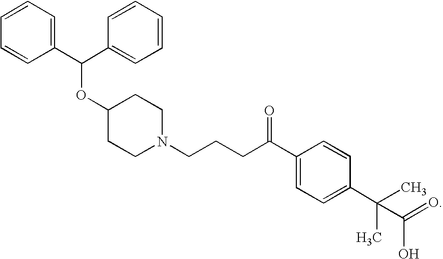

6. The method of claim 1, wherein a histamine H3 receptor antagonist is one or more members selected from the group consisting of thioperamide, impromidine, burimamide, clobenpropit, impentamine, mifetidine, clozapine, S-sopromidine, R-sopromidine, ciproxifam,

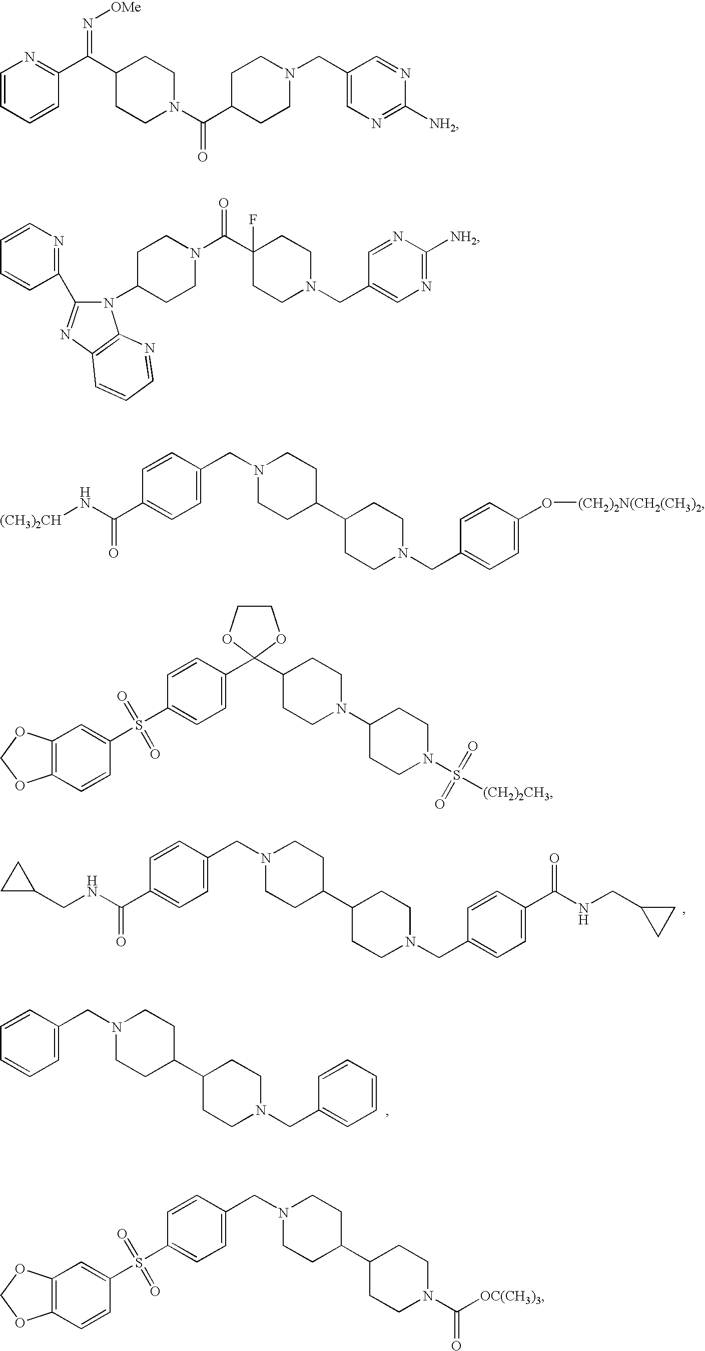

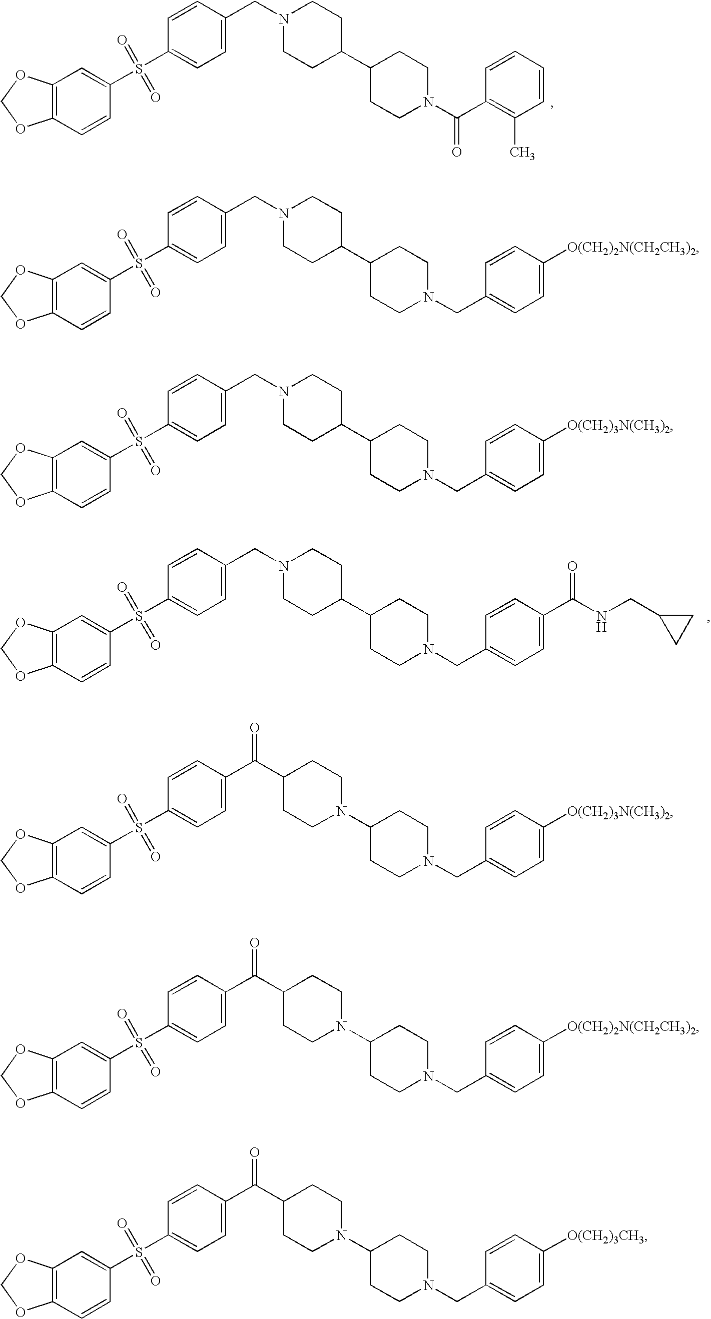

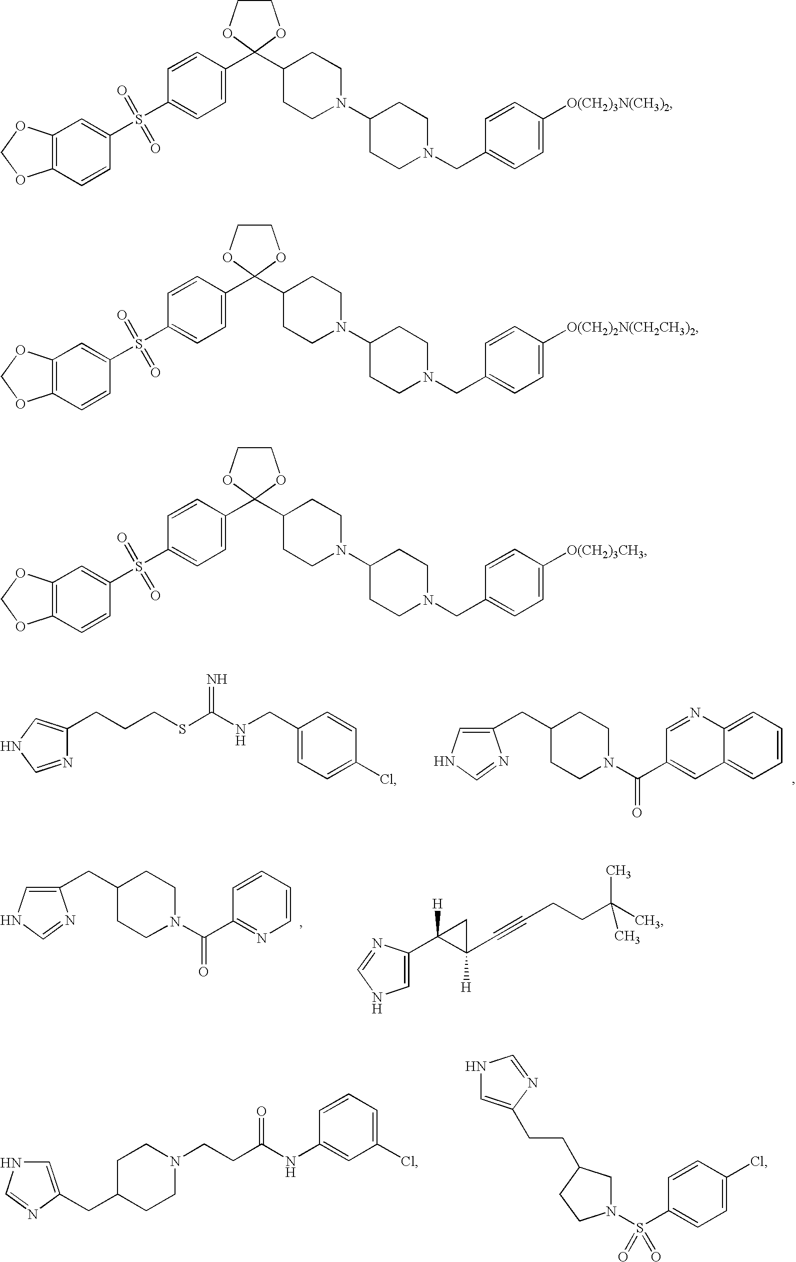

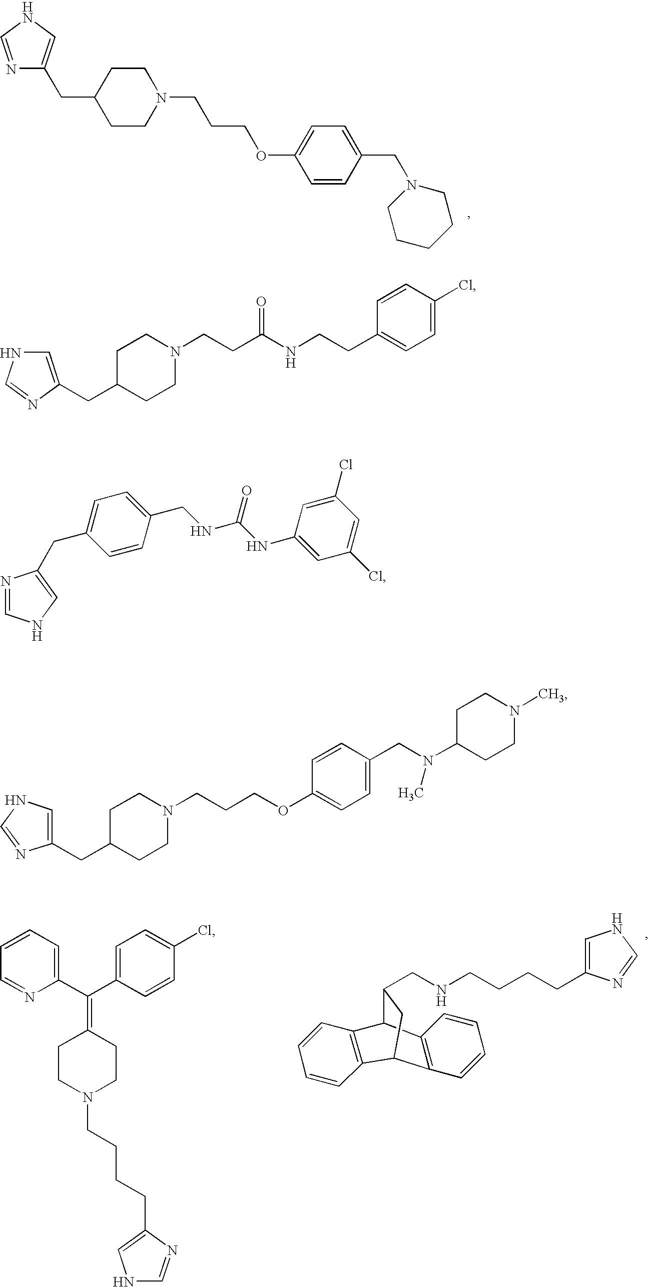

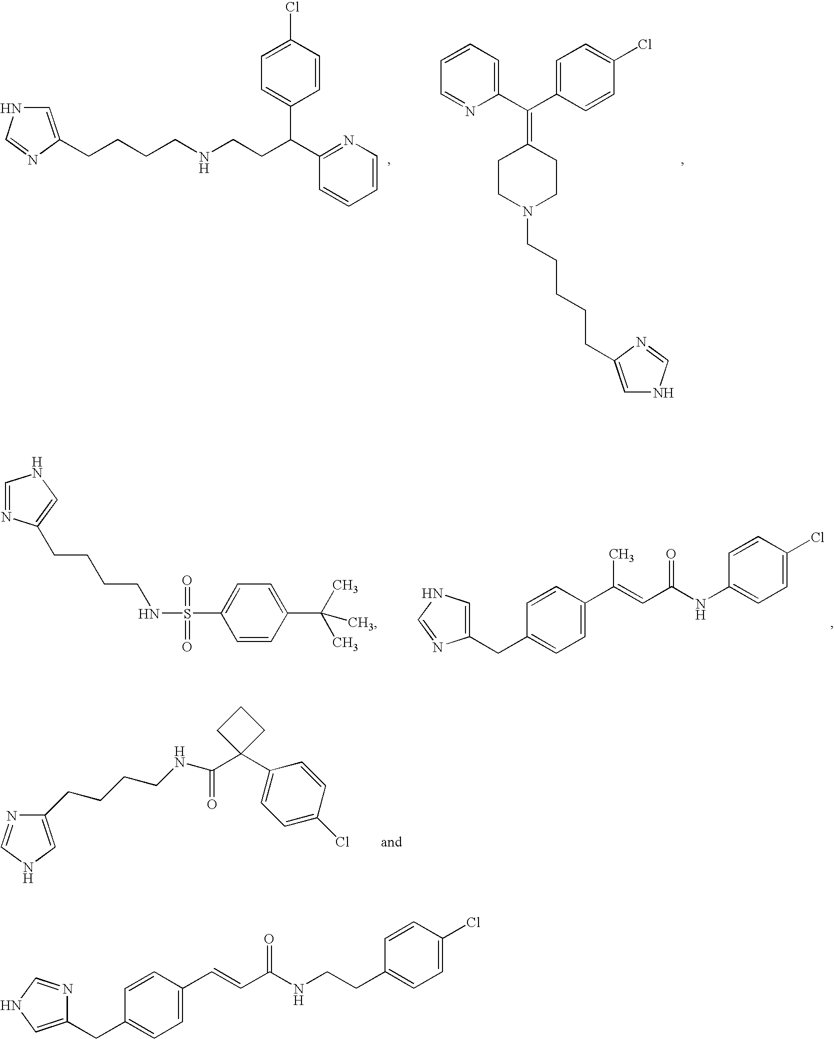

7. The method of claim 1 wherein the subject is administered chlorpheniramine and an antagonist selected from thioperamide and clobenpropit.

8. The method of claim 1 wherein a histamine H1 receptor antagonist and a histamine H3 receptor antagonist is administered to the subject parenterally.

9. The method of claim 1 wherein a histamine H1 receptor antagonist and a histamine H3 receptor antagonist is administered to the subject non-parenterally.

10. The method of claim 10 wherein a histamine H1 receptor antagonist and a histamine H3 receptor antagonist is administered to the subject topically.

11. The method of claim 1 wherein a histamine H1 receptor antagonist and a histamine H3 receptor antagonist are administered along with an additional agent selected from a non-steroidal anti-inflammatory drug, a steroid and an antibiotic.

12. The method of claim 1 wherein the subject is a human.

13. The method of claim 1 wherein the allergic skin condition is urticaria.

14. The method of claim 1 wherein the allergic ocular condition is selected from hay fever conjunctivitis, perennial allergic conjunctivitis, giant papillary conjunctivitis, vernal keratoconjunctivitis and atopic keratoconjunctivitis.

15. A combination comprising:
(a) one or more histamine H1 receptor antagonists; in association with
(b) one or more histamine H3 receptor antagonists.

16. The combination of claim 15 wherein a histamine H1 receptor antagonist is one or more members selected from the group consisting of astemizole, azatadine, azelastine, acrivastine, brompheniramine, chlorpheniramine, clemastine, cyclizine, carebastine, cyproheptadine, carbinoxamine, desloratadine, doxylamine, diphenhydramine, cetirizine, dimenhydrinate, dimethindene, ebastine, epinastine, efletirizine, fexofenadine, hydroxyzine, ketotifen, loratadine, levocabastine, mizolastine, mequitazine, mianserin, norebastine, meclizine, norastemizole, picumast, pyrilamine, promethazine, terfenadine, tripelennamine, temelastine, trimeprazine, triprolidine and

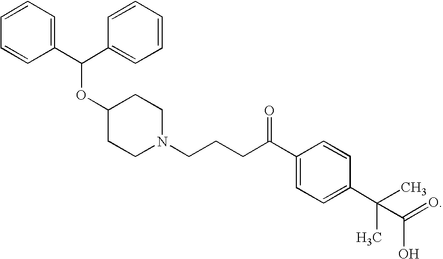

17. The combination of claim 15 wherein a histamine H1 receptor antagonist is one or more members selected from the group consisting of loratadine, desloratadine, chlorpheniramine, fexofenadine, promethazine, diphenhydramine and cetirizine.

18. The combination of claim 15 wherein a histamine H3 receptor antagonist is one or more members selected from the group consisting of thioperamide, impromidine, burimamide, clobenpropit, impentamine, mifetidine, clozapine, S-sopromidine, R-sopromidine, ciproxifam,

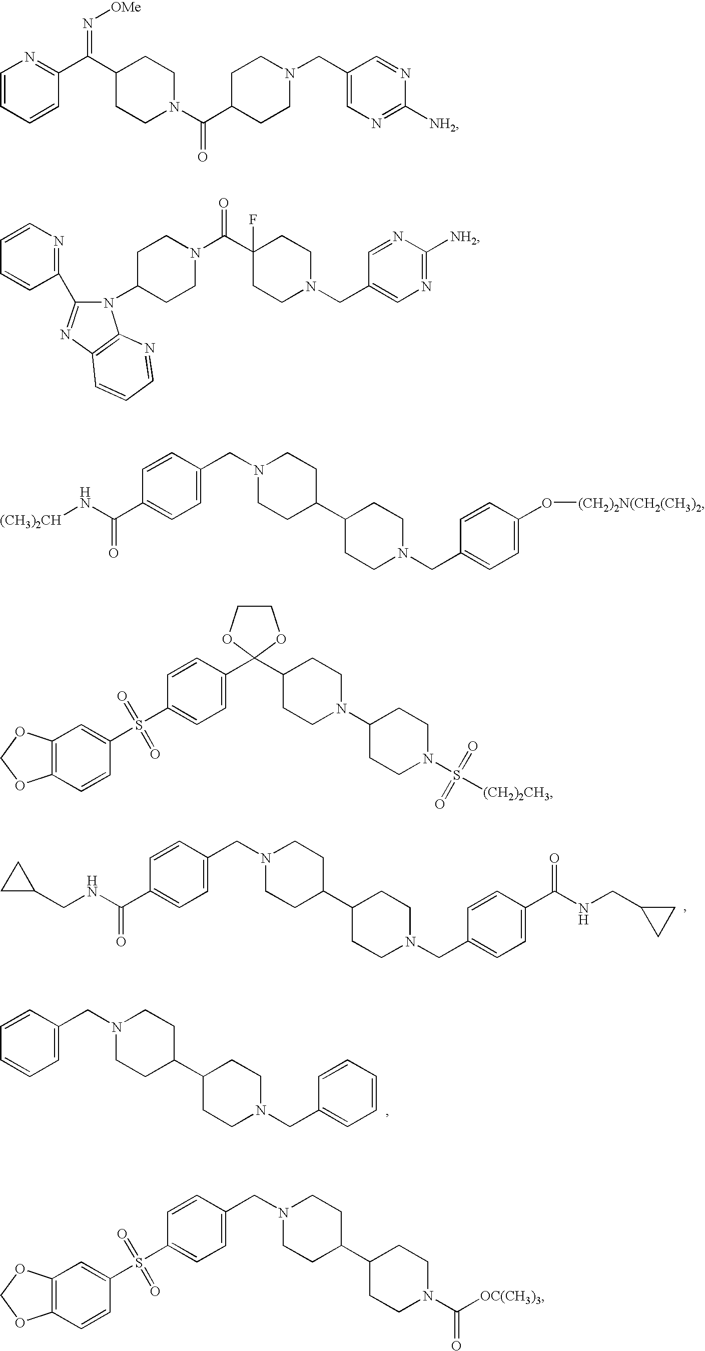

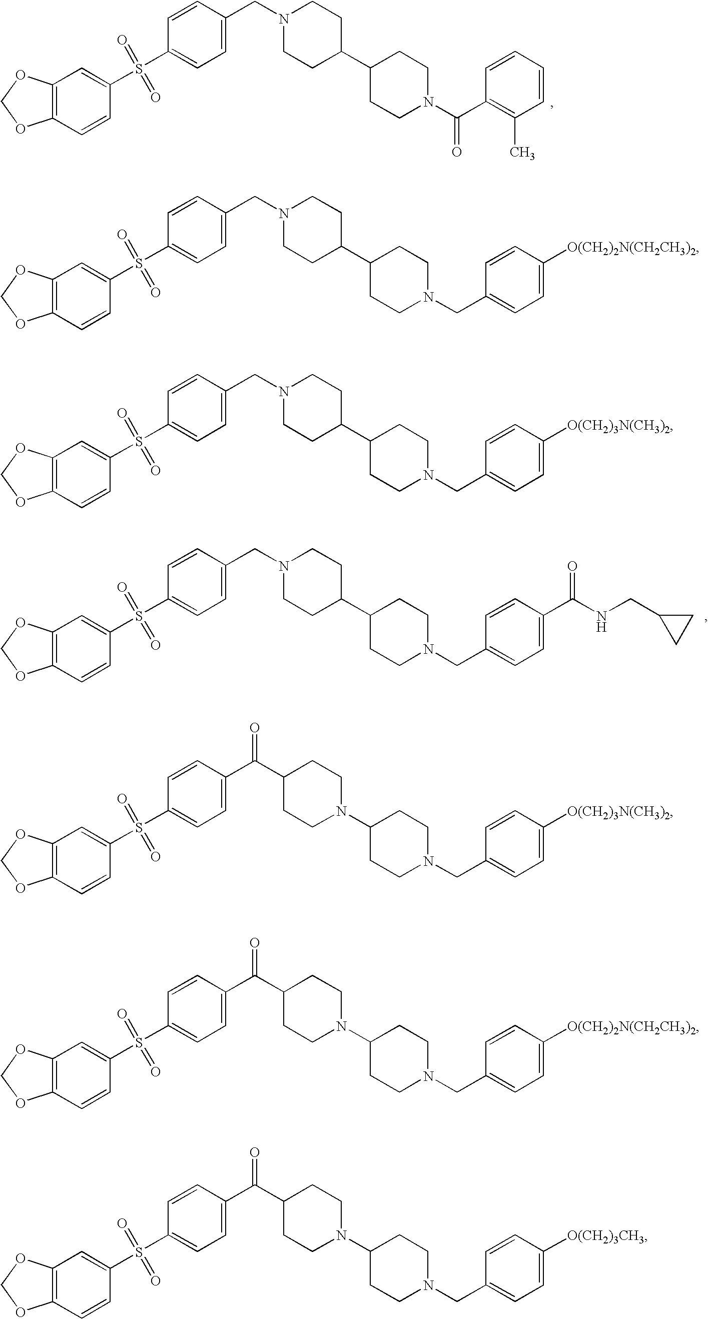

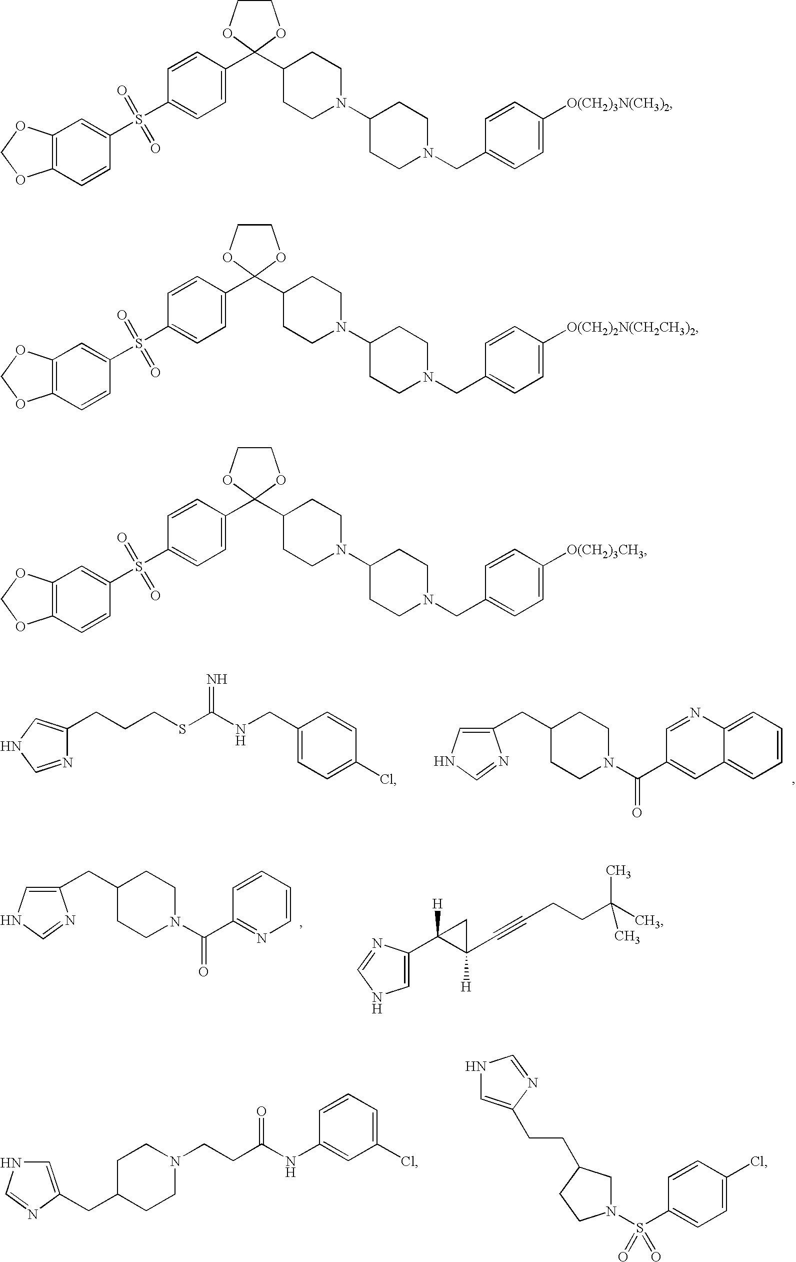

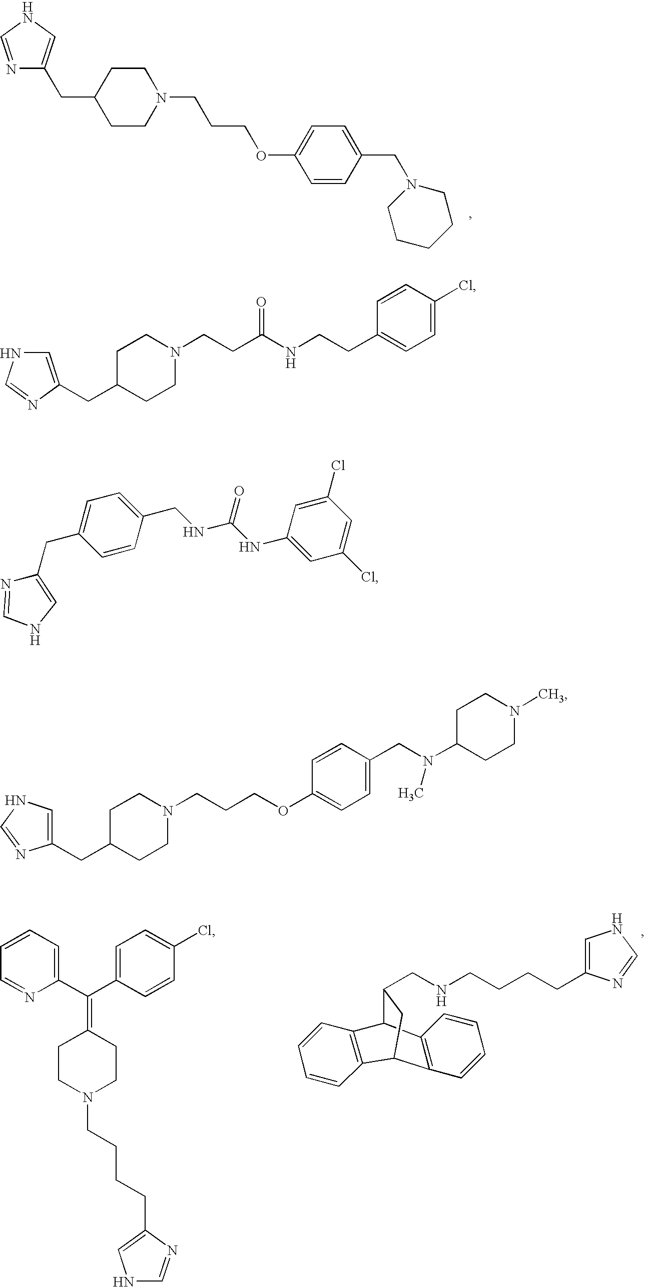

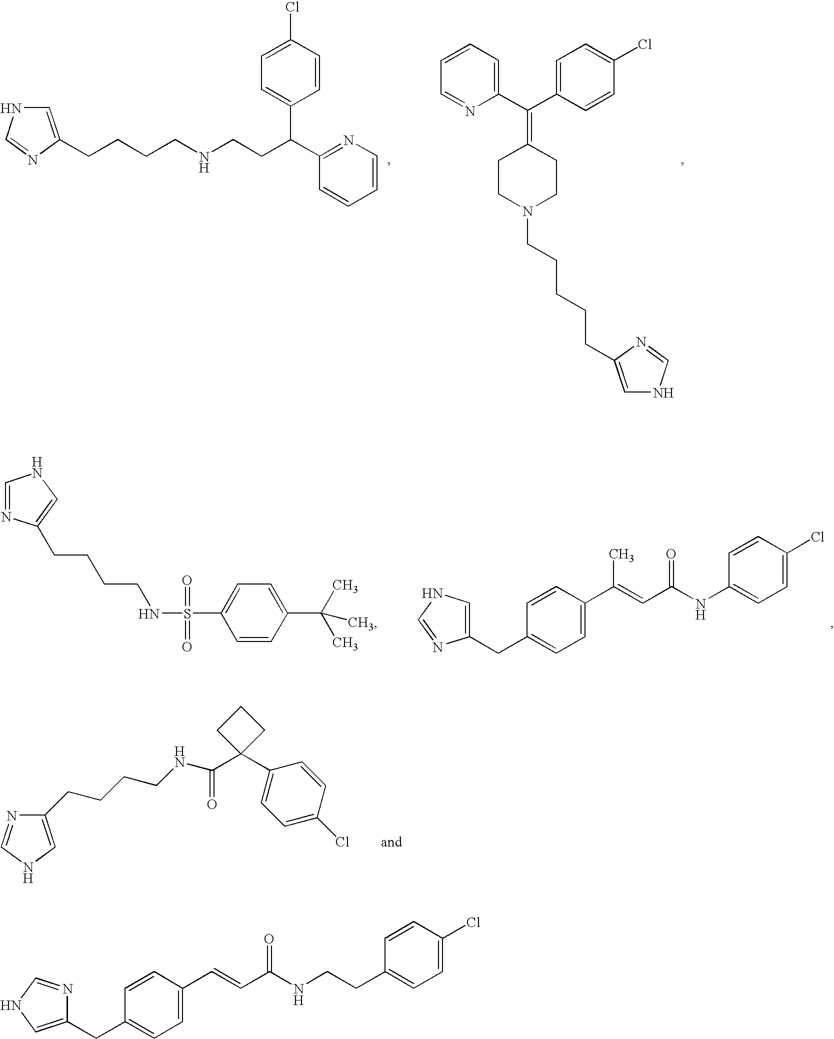

19. A pharmaceutical composition comprising a combination of claim 15 along with a pharmaceutically acceptable carrier.

20. The composition of claim 19 which is in the form of a pill, capsule or tablet.
